(19) United States
(12) Patent Application Publication
(10) Pub. No.: US 2005/0064545 A1
(43) Pub. Date: Mar. 24, 2005

(54) Recombinant protein expression

(76) Inventors: Ario DeMarco, Heidelberg (DE); Arie Geerlof, Hamburs (DE); Bernd Bukau, Heidelberg (DE); Elke Deuerling, Freiburg (DE)

Correspondence Address: ROTHWELL, FIGG, ERNST & MANBECK, P.C., 1425 K STREET, N.W., SUITE 800, WASHINGTON, DC, 20005, US

(21) Appl. No.: 10/500,883

(22) PCT Filed: Jan. 7, 2003
(86) PCT No.: PCT/IB03/00299

(30) Foreign Application Priority Data
Jan. 7, 2002 (GB) 0200250.9
Apr. 19, 2002 (GB) 0209013.2

(51) Int. Cl. C07H 21/04; C07K 14/705

(52) U.S. Cl. 435/69.1; 435/320.1; 435/325; 530/350; 536/23.5

(57) ABSTRACT

There are provided methods for the expression of a recombinant protein of interest, said methods comprising, in additional to various additional steps: a) culturing a host cell which expresses: i) one or more genes encoding the recombinant protein(s) of interest; ii) at least two genes encoding proteins selected from the group consisting of the chaperone proteins GroEL, GRoES, Dnak, DnaJ, GRpe, ClpB and their homologs (for example, Hsp104, Ydj1 and Ssa1 in yeast); under conditions suitable for protein expression; and separating said recombinant protein of interest from the host cell culture. Also provided are methods for increasing the degree of refolding of a recombinant protein of interest by ading a composition containing a chaperone protein to a preparation of the recombinant protein of interest in vitro.

The invention relates to methods for increasing the yield of folded recombinant protein in host cells.

All publications, patents and patent applications cited herein are incorporated in full by reference.

The overproduction of recombinant proteins in cellular systems frequently results in misfolding of these proteins. The fates of the misfolded recombinant proteins differ. They may refold to the native state or be degraded by the proteolytic machinery of the cell or be deposited into biologically inactive large aggregates known as ‘inclusion bodies’.

The folding of proteins and the refolding of misfolded soluble and aggregated proteins is known to be mediated by a network of evolutionarily conserved protein molecules called chaperones (Hartl, F. U., Nature, 381, 571-580, (1996); Horwich, A. L., Brooks Low K., Fenton, W. A., Hirshfield, I. N. & Furtak, K., Cell 74, 909-917 (1993); Ellis, R. J. & Hemmingsen, S. M., TiBS, 14, 339-342, (1989); Bukau, B., Hesterkamp, T. & Luirink, J., Trends Cell Biol., 6, 480-486, (1996); Bukau, B., Deuerling, E., Pfund, C. & Craig, E. A., Cell, 101, 119-122, (2000)). Major chaperones include members of evolutionarily conserved protein families, including the Hsp60 family (which includes the bacterial chaperone GroEL), the Hsp70 family (which includes the bacterial chaperone DnaK), the Hsp100 family (which includes the bacterial chaperone ClpB), the Hsp90 family (which includes the bacterial chaperone HtpG), the bacterial Trigger factor family, and the small HSPs (which includes the bacterial proteins IbpA and IbpB).

Bacterial systems like the gram-negative bacterium Escherichia coli are a popular choice for the production of recombinant proteins. In E. coli, it is known that the DnaK and GroEL/ES chaperone systems assist the de novo folding of proteins (Hartl, F. U., Nature, 381, 571-580, (1996); Ewalt, K. L., Hendrick, J. P., Houry, W. A. & Hartl, F. U. Cell 90, 491-500 (1997); Bukau, B., Deuerling, E., Pfund, C. & Craig, E. A., Cell, 101, 119-122, (2000); Teter, S. A. et al., Cell, 97, 755-765, (1999); Bukau, B. & Horwich, A. L, Cell, 92, 351-366, (1998); Deuerling, E., Schulze-Specking, A., Tomoyasu, T., Mogk, A. & Bukau, B. Nature 400, 693-696 (1999)).

Furthermore, DnaK and its co-chaperones DnaJ and GrpE are presently considered to form the most efficient chaperone system for preventing the aggregation of misfolded proteins (Mogk, A. et al., EMBO J., 18, 6934-6949, (1999); Tomoyasu, T., Mogk, A., Langen, H., Goloubinoff, P. & Bukau, B., Mol, Microbiol., 40, 397-413, (2001); Gragerov, A. et al., Proc. Natl. Acad. Sci. U.S.A. 89, 10341-10344 (1992)). Increased levels of GroEL and its co-chaperone GroES have been shown to prevent the heat induced aggregation of proteins in cells deficient of other major chaperones (Tomoyasu, T., Mogk, A., Langen, H., Goloubinoff, P. & Bukau, B., Mol, Microbiol., 40, 397-413, (2001); Gragerov, A. et al., Proc. Natl. Acad Sci U.S.A. 89, 10341-10344 (1992)).

Moreover, the disaggregation of protein aggregates in E. coli using chaperones has been proven for many different cellular proteins in vivo (Mogk, A. et al., EMBO J., 18, 6934-6949, (1999)), as well as in vitro using thermolabile malate dehydrogenase (MDH) as a reporter enzyme (Goloubinoff, P., Mogk, A., Peres Ben Zvi, A., Tomoyasu, T. & Bukau, B., Proc. Natl. Acad Sci., USA 96, 13732-13737, (1999)). Protein disaggregation is achieved by a bi-chaperone system, consisting of ClpB and the DnaK system. Large aggregates of MDH could be resolubilised in vitro and MDH was refolded afterwards into its native structure. Importantly, only the combination of both chaperones is active in resolubilisation and refolding of aggregated proteins. A recent publication showed that the resolubilisation of recombinant proteins from aggregates in vivo is possible. In these experiments, protein aggregates were generated by temperature upshift, and the solubilisation and refolding of these proteins was measured in the presence of protein synthesis inhibitors to ensure that only the pre-existing aggregated proteins were monitored. Molecular chaperones were able to resolve the aggregates under these conditions.

Previous studies also indicate that the solubility and yield of recombinant proteins could be enhanced by the overproduction of chaperones. Co-overproduction of GroEL/GroES enhanced the solubility of several recombinant proteins synthesised in E. coli (human ORP150, human lysozyme, p50^{csk} protein tyrosine kinase, phosphomannose isomerase, artificial fusion protein PreS2-S′-β-galactosidase) (Amrein, K. E. et al., Proc. Natl. Acad Sci., USA 92, 1048-1052 (1995); Nishihara, K., Kanemori, M., Yanagi, H. & Yura, T., Appl. Environ. Microbiol., 66, 884-889 (2000); Thomas, J. G. & Baneyx, F., Mol. Microbiol. 21, 1185-1196 (1996); Proudfoot, A. E., Goffin, L., Payton, M. A., Wells, T. N. & Bernard, A. R. Biochem J 318, 437442. (1996); Dale, G. E., Schonfeld, H. J., Langen, H. & Stieger, M., Protein Engineering, 7, 925-931 (1994)). The overproduction of the DnaK system together with recombinant target proteins elevates the solubility of endostatin, human ORP150, transglutaminase and the fusion protein PreS2-S′-β-galactosidase (Nishihara, K., Kanemori, M., Yanagi, H. & Yura, T., Appl. Environ. Microbiol., 66, 884-889, (2000); Thomas, J. G. & Baneyx, F., J Biol Chem 271, 11141-11147 (1996); Yokoyama, K., Kikuchi, Y. & Yasueda, H., Biosci. Biotechnol. Biochem. 62, 1205-1210 (1998)).

So far, no systematic approach has been made, to analyse whether the combination of all three chaperones systems (DnaK, DnaJ GrpE; GroES, GroEL and ClpB) expressed together with target genes in E. coil cells enhances solubility of recombinant proteins. Furthermore, none of the above-described studies allows the widespread optimisation of expression systems that is required to improve yields of soluble proteins on a general level. For example, each of the prior investigations focused on only one or a very small number of target proteins. These investigations also focused on the use of only one or two combined chaperone systems. In addition, none of these investigations addressed the issue of the importance of the ratio of the chaperones to one another and to the recombinant target protein. The previous studies therefore did not provide any understanding of the relationship between different chaperone proteins with respect to the folding/refolding of recombinant target proteins.

Accordingly, there remains a great need in the art for a general method to improve the yield of soluble recombinant protein in a given expression system. Such a method would allow the optimisation of expression systems to give maximal yields of soluble target proteins, and be of obvious industrial and commercial benefit.

The present invention is based upon the systematic engineering of cells for the controlled co-overexpression of different combinations of chaperone genes and target genes. In addition, it was investigated whether in vivo disaggregation and refolding of recombinant proteins from aggregates/inclusion bodies could be stimulated by enhanced levels of chaperones when the production of the target protein is stopped. As a result, the invention provides novel methods of optimising a given expression system in order to achieve higher yields of the desired soluble recombinant protein.

According a first aspect of the present invention, there is provided a method for the expression of a recombinant protein of interest, said method comprising:
a) culturing a host cell which expresses:
   i) one or more genes encoding one or more recombinant protein(s) of interest;
   ii) at least two genes encoding proteins selected from the group consisting of the chaperone proteins GroEL, GroES, DnaK, DnaJ, GrpE, ClpB and their homologs (for example, Hsp104, Ydj1 and Ssa1 in yeast); under conditions suitable for protein expression; and
b) separating said recombinant protein of interest from the host cell culture.

Through the recombinant engineering of host cells in this manner, the invention provides novel methods for producing a recombinant protein of interest, which have been found to lead to significant improvements in the levels of protein produced in the system. The mechanism is thought to be through increasing the folding rates of particular proteins using the co-expression of particular chaperones in controlled amounts. Using this system, very high yields of the desired soluble recombinant proteins of interest can be obtained.

Any recombinant protein of interest may be produced using the system of the invention. Preferred examples of proteins of interest will be apparent to the skilled reader. Particularly preferred recombinant proteins are those for which it is desirable to produce a large amount, and those of commercial interest.

Furthermore, the invention is readily applicable to a wide range of known expression systems by alterations in the cell culture techniques employed. For example, anaerobic fermenter-based cell culture would be appropriate for the culture of obligate anaerobes, whereas standard aerobic cell culture techniques would be appropriate for obligate aerobes. The nutrient composition of the culture medium may also be varied in accordance with the chosen expression system. The most suitable method of cell culture for a given expression system will be readily apparent to the skilled man.

Preferably, the genes selected in step a) ii) include DnaK, DnaJ and GrpE or homologs thereof, and may additionally include ClpB or a homolog thereof

In another preferred aspect of the invention, the genes selected in step a) ii) include GroES and GroEL or homologs thereof.

More preferably, the genes selected in step a) ii) include the DnaK, DnaJ, GrpE, ClpB, GroES and GroEL genes or homologs thereof.

The above combinations of chaperone proteins have been found to be particularly suitable for use in the methods according to the invention.

According to a further embodiment of the first aspect of the present invention, there is provided a method for the expression of a recombinant protein of interest, said method comprising:
a) culturing under conditions suitable for protein expression a host cell which expresses:
   i) one or more genes encoding one or more recombinant protein(s) of interest;
   ii) one or more genes encoding proteins selected from the group consisting of the chaperone proteins GroEL, GroES, DnaK, DnaJ, GrpE, ClpB and their homologs (for example, Hsp104, Ydj1 and Ssa1 in yeast);
   iii) one or more genes encoding proteins selected from the group consisting of the small heatshock proteins of the IbpA family and/or the IbpB family and/or their homologs; and
b) separating said recombinant protein of interest from the host cell culture.

The inclusion of a small heatshock protein of the IbpA family and/or the IbpB family with one or more of the chaperone proteins GroEL, GroES, DnaK, DnaJ, GrpE, ClpB in a host cell with a gene encoding a protein of interest has been shown to bestow significant beneficial effects on the level of expression of the recombinant protein.

For the purposes of this patent specification, two genes or proteins are said to be ‘homologs’ if one of the molecules has a high enough degree of sequence identity or similarity to the sequence of the other molecule to infer that the molecules have an equivalent function. ‘Identity’ indicates that at any particular position in the aligned sequences, the amino acid or nucleic acid residue is identical between the sequences. ‘Similarity’ indicates that, at any particular position in the aligned sequences, the amino acid residue or nucleic acid residue is of a similar type between the sequences. Degrees of identity and similarity can be readily calculated (Computational Molecular Biology, Lesk A. M., ed., Oxford University Press, New York, 1988; Biocomputing, Informatics and Genome Projects, Smith, D. W., ed., academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, New Jersey, 1987; and Sequence Analysis Primer, Gribskov, M. and Devereux, J., eds., M Stockton Press, New York, 1991).

The chaperone proteins for use in the invention therefore include natural biological variants (for example, allelic variants or geographical variations within the species from which the genes are derived) and mutants (such as mutants containing nucleic acid residue substitutions, insertions or deletions) of the genes. For the purposes of this application, greater than 40% identity between two polypeptides is considered to be an indication of functional equivalence. Preferred polypeptides have degrees of identity of greater than 70%, 80%, 90%, 95%, 98% or 99%, respectively. It is expected that any protein that finctions effectively as a chaperone, or as part of a chaperone system, within the host cells of the expression system will be of value in the described methods.

Preferably, the levels of the respective chaperone proteins are controlled in conjunction with the methods described above. Preferably, the levels of chaperone proteins are controlled by expressing the genes encoding the respective chaperone proteins from different promoters. Preferably, a selection or all of the promoters used are inducible. Different promoters may have different strengths and may respond to the same induction agent with different kinetics or be responsive to a different induction agent, allowing independent control of the expression level of each chaperone protein. Suitable promoters will be apparent to those of skill in the art and examples are given in standard textbooks, including Sambrook et al., 2001 (Molecular Cloning: A Laboratory Manual, Cold Spring Harbor Laboratory, Cold Spring Harbor, N.Y.); Ausubel et al., 1987-1995 (Current Protocols in Molecular Biology, Greene Publications and Wiley Interscience, New York, N.Y.). Examples of suitable promoters include IPTG-regulated promoters, such as the PA11 and lac-O1 promoters (see Tomoyasu, 2001).

Alternatively, or in addition, the respective chaperone proteins are expressed using expression systems of different strength. Examples of different expression systems will be clear to those of skill in the art; discussion of such systems may be found in standard textbooks, including Sambrook et al., 2001 (supra) and Ausubel et al., (supra). For example, the plasmid vector of the expression system may be a high copy number or low copy number plasmid. For instance, examples of E. coli compatible low copy number plasmids include pSC101 and p15A ori.

Preferably, the chaperone proteins are over-expressed relative to the expression levels that occur naturally in non-recombinant cells.

Similarly, the invention provides for the levels of the chaperone proteins relative to the recombinant protein(s) of interest to be controlled by expressing the genes encoding the respective proteins from different promoters, for the reasons described above. For example, in a system that utilises an IPTG-inducible promoter for expression of chaperone proteins, an arabinose-inducible promoter may be used to control expression of the recombinant protein of interest. In addition, the expression of the chaperones and of the recombinant proteins(s) can be controlled using different polymerases.

In a second aspect, the invention also provides methods comprising the use of a block in protein synthesis during the culturing steps a) described above. Preferably, the block in protein synthesis is imposed by addition of an effective amount of a protein synthesis inhibitor to the culture system, once a desired level of recombinant protein of interest has accumulated. More preferably, the chosen protein synthesis inhibitor is chloramphenicol, tetracycline, gentamycin or streptomycin. In order to ensure that protein synthesis is adequately inhibited, an effective amount of a protein synthesis inhibitor should be added. Details of effective amounts of protein synthesis inhibitor will be apparent to the skilled reader and are noted in standard textbooks. For example, for use in prokaryotic host cell systems, 200 μg/mL chloramphenicol is effective to inhibit protein synthesis.

Any other method that inhibits protein synthesis may also be of value for use with the methods of the invention. This includes the use of mutant strains that are conditionally defective in protein synthesis, for example because of the temperature sensitivity of an enzyme involved in plasmid or host cell DNA replication or in target gene and host gene transcription or in protein translation. The imposition of such a block in protein synthesis has been found to lead to significant increases in the level of recombinant protein that is generated in the system of the invention.

Alternatively, or in addition, the invention also provides for the use of a reduction in gene transcription, by removal of any agents that are effective to induce recombinant protein expression (such as IPTG for Lac repressor controlled genes), once a desired level of recombinant protein of interest has accumulated. Alternatively, a reduction of construct transcription could be achieved via the addition of a transcription blocking compound (such as glucose for catabolite repressable genes).

This aspect of the invention thus provides a method for the expression of a recombinant protein of interest, said method comprising:
a) culturing a host cell which expresses:
   i) one or more genes encoding one or more recombinant protein(s) of interest;
   ii) one or more genes encoding one or more proteins selected from the group consisting of the chaperone proteins GroEL, GroES, DnaK, DnaJ, GrpE, ClpB and their homologs (for example, Hsp104, Ydj1 and Ssa1 in yeast); under conditions suitable for protein expression;
b) imposing a block in protein synthesis, for example by addition of an effective amount of a protein synthesis inhibitor to the culture system, once a desired level of recombinant protein of interest has accumulated; and
c) separating said recombinant protein of interest from the host cell culture.

Also provided is a method for the expression of a recombinant protein of interest, said method comprising:
a) culturing a host cell which expresses:
   i) one or more genes encoding one or more recombinant protein(s) of interest;
   ii) one or more genes encoding one or more proteins selected from the group consisting of the chaperone proteins GroEL, GroES, DnaK, DnaJ, GrpE, ClpB and their homologs (for example, Hsp104, Ydj1 and Ssa1 in yeast); under conditions suitable for protein expression;
b) imposing a reduction in gene transcription, for example by removal of any agents that are effective to induce recombinant protein expression (such as IPTG for Lac repressor controlled genes), or via the addition of a transcription blocking compound (such as glucose for catabolite repressable genes), once a desired level of recombinant protein of interest has accumulated; and
c) separating said recombinant protein of interest from the host cell culture.

One or more genes encoding proteins selected from the group consisting of the small heatshock proteins of the IbpA family and/or the IbpB family and/or their homologs may also be included in the host cell. The inclusion of such proteins in conjunction with the imposition of a reduction in gene transcription or the imposition of a block in protien synthesis.

Preferably, a combination of chaperone proteins is expressed as described above.

Preferably, the chaperone proteins are expressed under a different promoter to that used to control expression of the recombinant protein of interest.

Preferably, the chosen protein synthesis inhibitor is chloramphenicol, tetracycline, gentamycin or streptomycin.

Preferably, in the methods of the above-described aspects of the invention the cultured host cell is a prokaryotic cell, such as an E. coil cell, a Lactococcus cell, a Lactobacillus cell or a Bacillus subtilis cell, or a eukaryotic cell such as a yeast cell, for example a Pichia or Saccharomyces yeast cell, or an insect cell, for example after baculoviral infection.

Preferably, an optimised yield of recombinant protein of interest is manifested by increasing the level of de novo protein folding.

An optimised yield of said recombinant protein of interest may also be manifested by increasing the level of in vivo refolding of aggregated, or misfolded soluble, recombinant protein.

An optimised yield of said recombinant protein of interest may also be manifested by increasing the level of in vitro refolding of aggregated, or misfolded soluble, recombinant protein.

An optimised yield of said recombinant protein may also be manifested by increasing the level of de novo protein folding in combination with increasing the increased level of in vivo refolding and/or in vitro protein refolding.

Preferably, said increased level of folding or refolding results in increased solubility of the recombinant protein of interest.

Preferably, said increased level of folding or refolding results in increased activity of the recombinant protein of interest.

According to a third aspect of the present invention there is also provided a method for increasing the degree of refolding of a recombinant protein of interest, said method comprising adding a composition containing a chaperone protein to a preparation of the recombinant protein of interest in vitro. This has been found to increase significantly the degree of refolding of protein in preparations containing wholly or partially unfolded protein. The preparation of the recombinant protein of interest may be any preparation that contains protein that is partially or wholly unfolded or misfolded. Preferably, the preparation is a cell extract preparation, such as a lysate of a prokaryotic cell.

Preferably, a combination of chaperone proteins as described above is added to the preparation of the recombinant protein of interest. For example, such chaperone proteins may include one or more genes encoding one or more proteins selected from the group consisting of the chaperone proteins GroEL, GroES, DnaK, DnaJ, GrpE, ClpB and their homologs (for example, Hsp104, Ydj1 and Ssa1 in yeast), and optionally one or more genes encoding proteins selected from the group consisting of the small heatshock proteins of the IbpA family and/or the IbpB family and/or their homologs.

The preparation of the recombinant protein of interest may be a preparation of soluble recombinant protein that has been precipitated in vivo, or may be a preparation of in vitro precipitated recombinant protein (for example, a host cell extract containing the recombinant protein aggregate).

Preferably, said composition containing the chaperone protein(s) is added after removal of any agents that are effective to induce soluble recombinant protein expression (such as IPTG for Lac repressor controlled genes) or after addition of a transcription blocking compound (such as glucose for catabolite repressable genes).

Preferably, the third aspect of the invention is used in conjunction with imposing a block in protein synthesis, for example by addition of an effective amount of a protein synthesis inhibitor to the culture system. As described above, chloramphenicol, tetracycline, gentamycin and streptomycin are examples of suitable protein synthesis inhibitors.

Preferably, when practising the above-described methods, the time course of refolding and the temperature at which refolding occurs is controlled. The time course of refolding and temperature at which it occurs are known to have a significant effect on the yield of soluble recombinant protein, and are thus an important aspect of a given expression system to be optimised for the maximal yield of soluble recombinant protein.

Preferably, when practising the above-described methods, a composition containing a protein selected from the group consisting of the small heatshock proteins of the IbpA family and/or the IbpB family and/or their homologs is used in conjunction with the chaperone proteins GroEL, GroES, DnaK, DnaJ, GrpE, and/or ClpB and/or their homologs.

A further aspect of the present invention relates to methods for the prophylaxis, therapy or treatment of diseases in which aggregated proteins are implicated, comprising the administration of the described combinations of chaperone proteins and/or small heatshock proteins in sufficient amounts. Such diseases include, but are not limited to diseases in which amyloid deposits are implicated, such as late and early onset Alzheimer's disease, SAA amyloidosis, hereditary Icelandic syndrome, multiple myeloma, and spongiform encephalopathies.

Various aspects and embodiments of the present invention will now be described in more detail by way of example. It will be appreciated that modification of detail may be made without departing from the scope of the invention.

### BRIEF DESCRIPTION OF THE FIGURES

FIG. 1A shows chaperone co-overproduction systems tested in E. coli. Genes encoding three different chaperone-systems (GroEL/ES; DnaK, DnaJ, GrpE; and ClpB) were cloned in a pair of low copy number vectors, which are compatible with E. coli (SC101 and p15A ori), carry the lacI^{Q} gene and different resistance markers for selection. Chaperone genes are set under the control of IPTG-regulated promoters (PA11/lacO1) for controlled expression. Each combination of vector pairs (1 to 5) differs in its combination and level of chaperone expression. In these strains subsequently a third plasmid encoding a substrate protein was introduced.

FIG. 1B shows chaperone expression patterns. The chaperone combinations 2 to 5 are shown. The left hand lane of each pair is loaded with a sample for which expression of the recombinant proteins had not been induced. The right hand column for each chaperone combination shows an IPTG-induced sample.

FIG. 2. Chaperone and target protein co-expression under IPTG control. The target proteins Tep4, Btke and Lzip were purified by metal affinity chromatography after transformation in BL21(DE3) cells used as a control (K) and in the same strain but co-expressing the 5 different chaperone combinations reported in FIG. 1.

FIG. 3. In vivo induced refolding. FIG. 3A shows the Btke expression level after chaperone-induced re-folding in BL21(DE3) cells used as a control (K) and in the same strain but co-expressing the 5 different chaperone combinations reported in FIG. 1. Cells were grown at 30° C., induced with 0.1 mM IPTG, grown overnight, and then either grown 2 more hours (first lane of each combination) or pelletted, re-suspended in fresh medium plus 200 μg/mL chloramphenicol and cultured 2 more hours (second lane). FIG. 3B shows optimisation of the re-folding conditions using the chaperone combination 4 shown in FIG. 3B. After overnight culture at 20° C. the cells were pelletted, resuspended in fresh medium and cultured 1 h, 2 h, 3 h, and 4 h at 20° C. (1 to 4), or 1 h and 2 h at 37° C. (5 and 6) in the presence of 200 μg/mL chloramphenicol. For each combination the first lane was loaded with the uninduced sample and the second with the treated one. FIG. 3C shows Btke expressed in control (C1) and chaperone combination 4 (C2) cells. Lanes were loaded with uninduced samples (K), induced and cultured at 20° C. overnight plus two hours at the same temperature, pelleted after overnight growth, resuspended in fresh medium plus 200 μg/mL chloramphenicol and cultured 2 more hours, as in 2 but in the presence of 1 mM IPTG instead of chloramphenicol, resuspended in fresh medium for 1 h, 2 h, and 4 h. The numbers shown below the gel image indicate the increase factor obtained comparing the intensity of the bands to the reference (induced cells without chaperone co-expression). FIG. 3D shows the effect of growth conditions on re-folding efficiency of Btke. Cells were grown overnight at 20° C. (D1) and at 42° C. before inducing the re-folding at 20° C. (D2). Lanes were loaded with un-induced samples (K), induced and cultured overnight plus two hours (1), resuspended in fresh medium plus 2 h culture (2), in fresh medium plus 200 μg/mL chloramphenicol and cultured 2 more hours (3). FIG. 3E shows the re-folding efficiency of Tep4 expressed in control (E1) and chaperone combination 4 (E2) cells. Lanes were loaded with uninduced samples (K), induced and cultured overnight plus two hours (1), resuspended in fresh medium plus 2 h culture (3), in fresh medium plus 200 μg/mL chloramphenicol and cultured 2 more hours (4).

FIG. 4. In vitro re-folding. FIG. 4A shows Btke expressed either in control cells (c) or in cells co-expressing chaperone combination 3 or 4. 3 h after IPTG induction, cells were harvested and lysate prepared as described above. Samples containing 100 μg lysate were supplemented with 10 mM ATP and 3 mM PEP and 20 ng/ml PK. After indicated timepoints, soluble Btke protein was isolated and analysed by SDS-PAGE and Coomassie staining. FIG. 4B shows the results produced when pellets with insoluble Btke were isolated from control cells. Pellets were suspended in buffer and where indicated chaperones were added. After 5 min, 2, 4, and 20 h soluble Btke protein was isolated as described above and analysed by SDS-PAGE and silver staining.

FIG. 5 shows the results of experiments to test the effects of various combinations of different sHSPs and HSPs on the refolding of soluble MDH complexes in vitro.

FIG. 6 shows the results of experiments to test the effects of different HSP combinations on the refolding of soluble α-glucosidase/sHSP 16.6 and citrate synthase/sHSP 16.6 complexes in vitro.

FIG. 7 shows the results of experiments to test the effects of different HSP combinations on the refolding of aggregated luciferase and soluble luciferase/sHSP 16.6 complexes in vitro

FIG. 8 shows the results of KJE/ClpB-mediated refolding of MDH. The different 16.6 concentrations present during MDH denaturation are shown as the indicated 16.6/MDH ratio. Refolding curves for KJE-mediated refolding of MDH are indicated. Refolding curves for refolding of MDH carried out in presence of ClpB/DnaK are differently coloured. The precise 16.6/MDH ratios during MDH denaturation are indicated to the right of the graph and are as follows: green (16.6/MDH ratio=0); light blue (16.6/MDH ratio=0.25); brown (16.6/MDH ratio=0.5); dark blue (16.6/MDH ratio=1); yellow (16.6/MDH ratio=2); pink (16.6/MDH ratio4).

FIG. 9 shows the results of experiments to determine the effect on protein refolding of varying the concentration of ClpB.

FIG. 10 shows the results of experiments to determine the effects of mutations to the ibpAB genes and DnaK genes of E. coli.

FIG. 11 shows a comparison between the effects of mutations to the ibpAB and clpB genes in E coli on the thermotolerance of those strains.

FIG. 12 shows the results of experiments to determine whether IbpA/B protein function increases in importance in the presence of reduced levels of DnaK and at elevated temperatures.

FIG. 13 shows the results of experiments to determine the levels of protein aggregation associated with heat shock in ΔibpAB ΔclpB double knockout E. coli cells.

FIG. 14 shows the effect of IpbAB co-expression on the level of soluble target proteins produced in E. coli cells.

FIG. 15: Effect of plasmid interactions on the level of the recombinant protein expression. A) Recombinant chaperone (K-DnaK, ELS-GroELS, ClpB) accumulation in bacteria homogenates. B) Accumulation of co-expressed recombinant chaperones and target protein GTR1 in the homogenates recovered from control (C) and induced (I) bacteria. C) Effect of chaperone co-transformation on the not induced (C) and IPTG-induced (I) expression of the target protein Btk cloned in pET24d. D) Effect of the co-transformation with an empty pDM1 vector on the not induced expression of Btk cloned in pET24d.

FIG. 16: Co-expression of the coil-coiled region of Xklp3A/B. The chains A and B were cloned in a polycistronic vector and expressed either in BL21 (DE3) together with the recombinant chaperone combination K+J+E+ClpB+GroELS (+chap) or in BL21 (DE3) pLysS in the presence of 1% glucose (−chap).

FIG. 17: Effect of unsynchronised recombinant chaperone expression on the level of soluble target recombinant protein. The independent induction of the chaperones and target proteins has been obtained using arabinose-regulated vectors for the target proteins and IPTG-inducible vectors for the chaperones. In the figures are reported the bands corresponding to the soluble target protein purified by affinity chromatography from 0.5 20 mL of bacterial culture. A) Amount of soluble GTR1 and coiled-coil Xklp3A recovered from wild type cells and bacteria co-transformed with different chaperone combinations. Expression was induced by 0.2 mM IPTG and 1.5 mg/mL arabinose were added 20 min later. The samples were collected 3 hours after the IPTG induction. B) Amount of soluble GTR1 recovered from bacteria co-transformed with K+J+E+ClpB+GroELS and using different combinations of time and expression-inducer concentrations. The samples were collected 3 hours after the addition of the first inducer and the bands corresponding to GroEL are recovered from SDS-gels loaded with the soluble fraction after cell lysis. C) Amount of soluble coiled-coil Xklp3B recovered from bacteria co-transformed with K+J+E+ClpB+GroEL after overnight culture (ON) at 20° C. The replacement of the ON medium with fresh medium (Fr. Md.), 0.2 mM chloramphenicol (Chlor.) and the temperature shift to 30° C. were used to stimulate the in vivo re-folding of the aggregated target protein.

### EXAMPLES

Examples 1-5 below illustrate the materials and methods used to investigate the effect of co-expressing different chaperone combinations on the yield of a large variety of different recombinant proteins.

### Example 1

### Construction of Chaperone Vectors

Plasmids carrying chaperone genes under the control of the IPTG-sensitive promoter PA1/lacO-1 were constructed as described (Tomoyasu, T., Mogk, A., Langen, H., Goloubinoff, P., Buckau, B., Mol. Microbiol., 40, 397-413, (2001)). Target protein vectors were delivered to the Protein Expression Unit from different research groups working at the European Molecular Biology Laboratory.

### Example 2

### Transformation Procedure

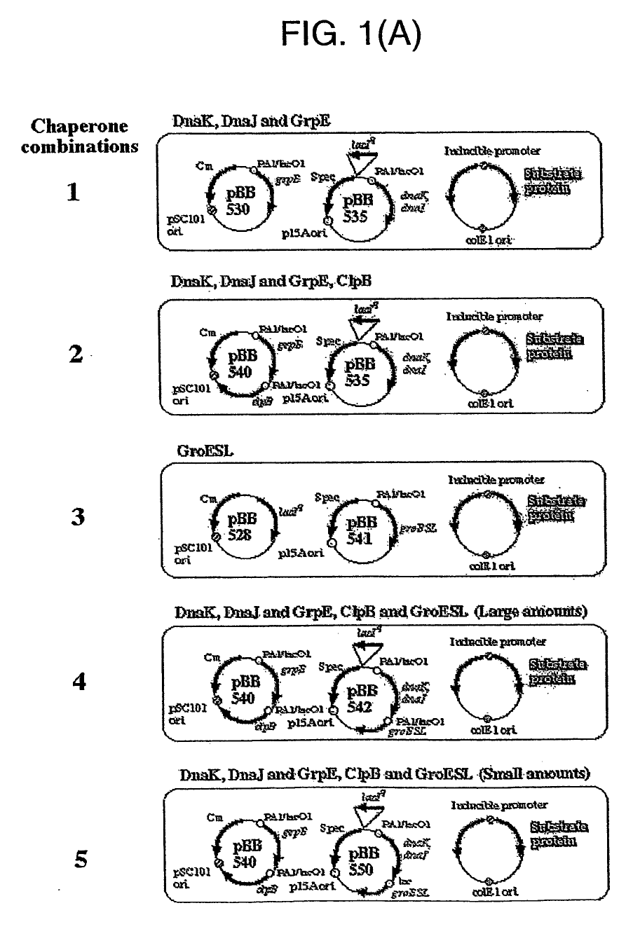

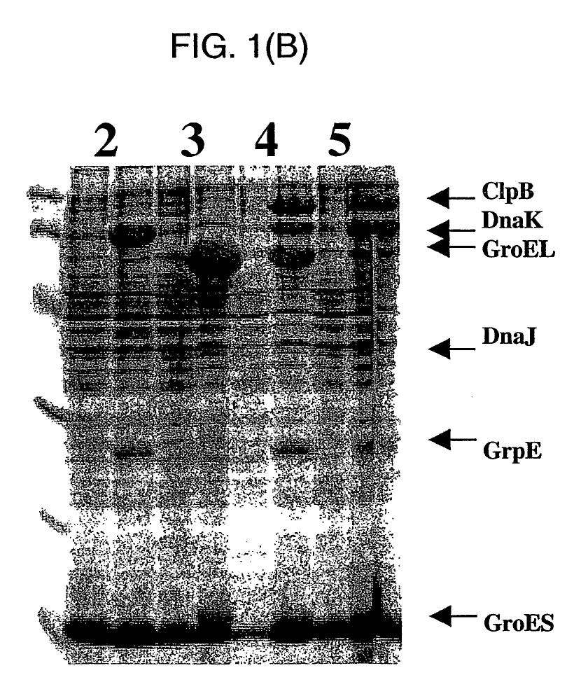

Competent BL21 (DE3) and Top10 cells were transformed with the following couples of plasmids for selective expression of chaperone combinations (FIG. 1A). The clones for DnaK, DnaJ, and GrpE were carried by pBB530 and pBB535; the co-expression of DnaK, DnaJ, GrpE, and ClpB was regulated by pBB535 and pBB540; GroEL/ES system was expressed by pBB528 and pBB541; a large amount of the complete system DnaK, DnaJ, GrpE, ClpB, and GroEL/ES was ensured by pBB540 and pBB542; finally, a lower expression level of the same chaperone combination was obtained using pBB540 and pBB550. A complete array of single chaperone plasmid transformed cells was also prepared as a control. Transformed cells were checked for chaperone expression and successively made competent. The protease deficient strain BB7333 (MC4100 ΔclpX, ΔclpP, Δlon) was used for transforming the Btkp protein. These strains were also made competent and used for a further transformation with the target proteins.

### Example 3

### Cell Cultures

Single colonies from the transformed cells were used to inject 3 mL of LB medium. Liquid cultures were performed initially at 37° C., then transferred to 30° C. and finally transferred to 20° C. Using different times of incubation at the higher temperatures it was possible to reach the OD₆₀₀ of 0.8 at the same time for all the different cell strains cultured together for comparative expression assays. Protein expression was performed overnight by inducing gene transcription using 0.1 mM IPTG. 1.5 mL of the overnight culture of both IPTG-induced (hereafter termed ‘induced) and control bacteria was directly centrifuged in an Eppendorf tube and the pellet frozen and stored at −20° C. Alternatively, the pellet was re-suspended in 3 mL of fresh medium and divided into two aliquots of 1.5 mL, with or without the addition of 200 μg/mL chloramphenicol. After 2 h culture at 20° C. the cells were harvested as described before. Inclusion body overproduction was obtained by culturing the bacteria at 42° C. overnight after induction. Large scale cultures were grown in 2 L flasks using 5 mL of overnight LB pre-culture to inoculate 500 mL of Terrific Broth.

### Example 4

### Protein Purification and Evaluation

Frozen bacterial pellets were re-suspended in 350 μL of 20 mM Tris HCl, pH 8.0, 2 mM PMSF, 0.05% Triton X-100, 1 μg/mL DNAase and 1 mg/mL lysozyme and incubated on ice for 30 min, with periodic stiring. The suspension was sonicated in water for 5 minutes, an aliquot (of homogenate) was stored and the rest was pelleted in a minifuge. An aliquot of the supernatant was preserved and the rest was added to 15 μL of pre-washed magnetic beads (Qiagen) and incubated further 30 min under agitation before being removed. Beads were washed 30 min with 20 mM K-phosphate buffer, pH 7.8, 300 mM NaCl, 20 mM imidazole, 8% glycerol, 0.2% Triton X-100 and later with PBS buffer plus 0.05% Triton X-100. Finally they were boiled in 12 μL SDS sample buffer and the samples loaded for SDS PAGE analysis, using a Pharmacia minigel system. Proteins were detected after coloration with Simply Blue Safestain (Invitrogen) following the manufacturer's instructions and the gels were recorded using a Umax Astra 4000U scanner. Bands corresponding to the proteins were analysed using the public NIH Image 1.62f software. Alternatively, protein was eluted from washed beads using 30 μL PBS buffer plus 0.5M imidazole and its relative concentration measured following its adsorbance at 280 nm. The proper folding was evaluated by circular dichroism using a J-710 spectropolarimeter (Jasco).

### Example 5

### In vitro Experiments

Cells were grown in LB and after inducing the synthesis of either Btke or Btke together with GroEL/ES (combination 3) or together with GroEL/ES, DnaK, DnaJ, GrpE, ClpB (combination 4) for 3 h with 1 mM IPTG at 37° C., lysates were prepared as described above. For refolding of Btke from inclusion bodies using total lysate, 10 mM ATP, 3 mM phosphoenole pyruvate (PEP) and 20 ng/ml pyruvate kinase (PK) were added and incubated at 20° C. After 5 min, 2, 4, and 20 h soluble material was separated from insoluble fractions by centrifugation (15 min, 4° C., 10.0000 rpm) and the soluble fraction was used to isolate target protein as described above.

For resolubilsation of isolated Btke aggregates with exogenous chaperone addition, 100 μg of total lysate (isolated from cells with overproduced Btke) was centrifuged for 15 min and pellets were resuspended in 20 mM Tris/HCl, 100 mM KCl and 20 mM MgCl. Chaperones were added as indicated and samples incubated at 20° C. for 5 min, 2, 4, 20 h. Soluble material was separated from inclusion bodies by centrifugation and isolated as described.

Examples 6 to 9 below illustrate the optimisation of chaperone co-expression combinations and other experimental variables in order to greatly increase the yield of a large number of diverse recombinant proteins.

### Example 6

### Investigation of the Effect of Chaperone Combinations on de novo Protein Folding

Five different combinations of plasmids encoding chaperone systems (GroEL/ES; DnaK, DnaJ, GrpE and ClpB) in different combinations and amounts under the control of IPTG regulated promoters were introduced into BL21 (DE3) cells as illustrated in FIG. 1A. The degree of chaperone expression was shown to be very high (FIG. 1B). These cells were subsequently transformed with plasmids expressing substrate proteins in an IPTG controlled manner (FIG. 1A). Therefore, co-expression of chaperones and target proteins was obtained by simultaneous induction of all the promoters with IPTG. Co-expression of chaperones together with 50 different target genes was tested. For each target protein, all five different chaperone combinations were tested and solubility of the recombinant proteins analysed. In summary a higher yield of soluble substrate protein was achieved in more than 50% of the tested constructs (see Table 1 below).

Table 1 shows a list of the proteins used in the survey for analysing the effect of chaperone co-expression on soluble target protein yield. The table shows the molecular weight of the constructs, the original organisms from which they were cloned, whether they corresponded to full length proteins (F1) or to domains, expressed alone or fused to a partner (fus), and their cell localisation (cytoplasm, membrane, nucleus, secreted) in vivo. The yield increase factor (IF) induced by the best chaperone combination is reported under ‘Chap. IF’ and the yield increase factor obtained using the refolding protocol under ‘Refolding IF’. The symbol (/) signifies that the experiment has not yet been done and (!) that protein has been obtained using constructs that gave no soluble protein when expressed in wild type bacteria.

TABLE 1

| Protein | MW | | Organism | Features | Chap. IF | Refolding IF |
|---|---|---|---|---|---|---|
| GTR1 | 40 | kD | S. cerevisiae | Fl/cyt | 3 | 3 |
| BtKp | 55 | kD | H. sapiens | domain/cyt | 0 | 28 |
| Xpot1 | 110 | kD | H. sapiens | Fl/cyt | 0 | / |
| XklpA1 | 62 | kD | X. laevis | domain/fus/cyt | 0 | ! |
| XklpB1 | 40 | kD | X. laevis | domain/fus/cyt | 0 | ! |
| HbpH | 9 | kD | H. sapiens | domain/cyt | 3.5 | 3.5 |
| TEVprotease | 30 | kD | TEV | domain | 3.5 | / |
| Pex5p | 50 | kD | H. sapiens | domain/cyt | 0 | / |
| UCP1 | 33 | kD | R. norvegicus | domain/membr | 0 | 0 |
| Transcr Fact | 37 | kD | H. sapiens | Fl/cyt | 0 | 0 |
| BtKe | 55 | kD | H. sapiens | domain/cyt | 4 | 42 |
| XklpA2 | 38 | kD | X. laevis | domain/fus/cyt | 0 | / |
| XklpB2 | 35 | kD | X. laevis | domain/fus/cyt | 0 | / |
| XklpA3 | 72 | kD | X. laevis | domain/fus/cyt | 0 | / |
| Rolled | 43 | kD | D. melanogaster | Fl/cyt | 4.5 | 4.5 |
| Lzip | 41 | kD | H. sapiens | Fl/cyt | ! | / |
| 1Ap | 52 | kD | D. melanogaster | Fl/nucl | 0 | / |
| Chip | 64 | kD | D. melanogaster | Fl/nucl | 0 | / |
| dLMO | 37 | kD | D. melanogaster | Fl/nucl | 0 | / |
| Tlc | 57 | kD | R. prowazekii | Fl/membr | 0 | / |
| BtKc | 64 | kD | H. sapiens | Fl/cyt | 3 | / |
| PhosphK | 29 | kD | H. sapiens | Fl/cyt | 3 | 7 |
| Compl. Tep3 | 47 | kD | A. gambiae | domain/fus | 4 | / |
| Compl. Tep4 | 45 | kD | A. gambiae | domain/fus | 3.5 | / |
| XklpA4 | 72 | kD | X. laevis | domain/fus/cyt | 2.5 | 2.5 |
| XklpB3 | 73 | kD | X. laevis | domain/fus/cyt | 2.5 | 2.5 |
| E8R1 | 58 | kD | Vaccinia virus | Fl/membr/fus | 7 | / |
| Compl. Tep3 | 70 | kD | A. gambiae | domain/fus/secr | 0 | 11 |
| Compl. Tep4 | 68 | kD | A. gambiae | domain/fus/secr | 3.5 | 13 |
| MaxF | 7.5 | kD | syntetic | domain | 3 | / |
| XklpA5 | 35 | kD | X. laevis | domain/fus/cyt | 0 | 19 |
| E8R2 | 85 | kD | Vaccinia virus | Fl/membr/fus | 5.5 | 5.5 |
| Susy | 90 | kD | Z. mays | Fl/membrane | 3 | 5 |
| Mash | 91 | kD | Z. mays | Fl/cyt | 0 | 3 |
| PPAT | 22 | kD | E. coli | Fl/cyt | 0 | 3 |
| 2Ap | 54 | kD | D. melanogaster | Fl/nucl | ! | 3 |
| F10L | 45 | kD | Vaccinia virus | Fl/fus | 0 | 0 |
| B1R | 47 | kD | Vaccinia virus | Fl/fus | 3.5 | 3.5 |
| 1Frenge | 43 | kD | D. melanogaster | domain/cyt | ! | ! |
| Tep1 | 7 | kD | A. gambiae | domain/secr | 3 | 6 |
| Tep2 | 11 | kD | A. gambiae | domain/secr | 0 | 0 |
| 2Frenge | 55 | kD | D. melanogaster | domain/fus | 0 | 2 |
| GFP-fusion | 95 | kD | A. victoria | Fl/fus/cyt | 0 | 0 |
| 2C18 | 50 | kD | H. sapiens | Fl/fus | 3 | 8 |
| 22j21 | 72 | kD | H. sapiens | Fl/fus | ! | ! |
| XklpA + B | 15 + 17 | kD | X. laevis | domain/complex | 2.5 | 3.5 |
| Msl3 | 14 | kD | D. melanogaster | domain/cyt | 2.5 | 2.5 |
| Mash + Susy | 94 + 90 | kD | Z. mays | Fl/complex | 3 | 3 |
| Endostatin | 22 | kD | M. musculus | domain/secr | 0 | 0 |
| Kringle | 30 | kD | H. sapiens | domain/fus | 0 | 0 |

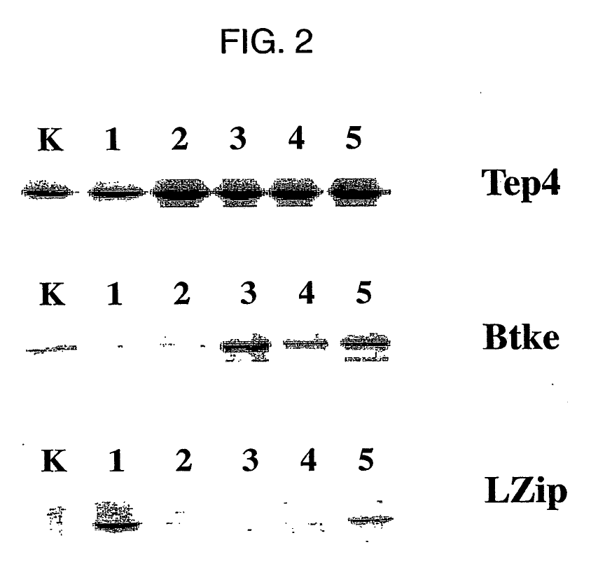

As can be seen from the ‘Chap. IF’ ratings, soluble target protein yield increased between 2.5 and 7-fold. Effects of co-expressed chaperones were not limited to a certain type of substrate protein. The target proteins tested were representative of several different classes, including complexes, soluble, membrane-bound and secreted proteins, full-length, domains and fusion constructs, with a molecular weight spanning from 7.5 to 110 kD, expressed in the cytoplasm and in the periplasm (Table 1). Moreover, in some cases, like Lzip (see Table 1 and also FIG. 2), co-expression of chaperones was the only possibility to obtain any soluble protein. Evaluation of the 23 positive cases indicated that the most efficient chaperone combination was the fourth, which expressed all three chaperone systems in large amounts, followed by the third, fifth, first and the second. Nevertheless, as is demonstrated in the case of Lzip transcription factor where chaperone combination 1 worked far better than the others, any one chaperone combination is not necessarily optimal for all target proteins. Thus, despite the systematic approach it was not possible to infer general rules about the optimal conditions to succeed. No protein class showed better results in combination with particular chaperone combinations and no expression vector ensured significantly better yields. The only exception was when target proteins were cloned in high copy number vectors. In such a case no positive result was observed. The competition for the protein synthesis machinery could be considered as a reason, since chaperone expression is inhibited when a target protein was co-expressed and is completely prevented in cells harbouring expression vectors with pUC origin (data not shown). The results shown in Table 1 clearly demonstrate the very large increases in yield possible via the use of the disclosed methods.

### Example 7

### Testing the Effect of Co-overexpression of Chaperone Combinations and Target Proteins on Re-folding of Aggregated Proteins Using Chloramphenicol

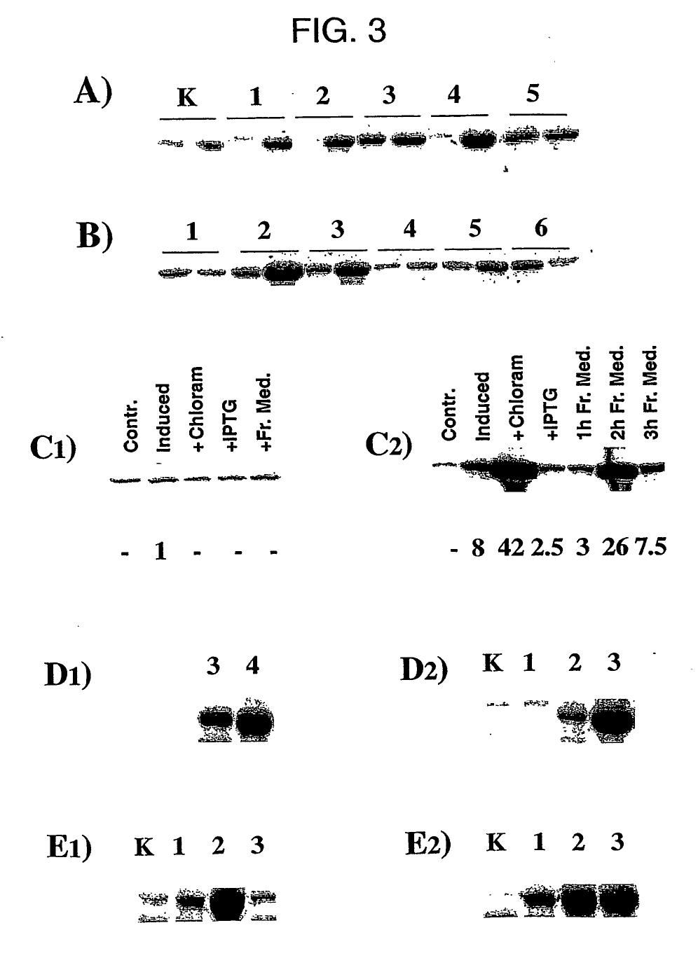

In the experiments of Example 5 it was often observed that inclusion bodies accumulated even in the presence of overproduced chaperones increasing the amount of soluble proteins. A recent paper (Carrio, M. M. and Villaverde, A. FEBS Lett., 489, 29-33 (2001)) showed that soluble proteins could be recovered in vivo from inclusion bodies when the protein synthesis was blocked by chloramphenicol addition and the whole cellular folding machinery became available for precipitated proteins. Therefore, we investigated the overexpression of chaperones not only for keeping recombinant proteins soluble but also for increasing the re-folding capability of cells. To investigate this further, we co-overexpressed chaperones and target genes as described before. Subsequently, we stopped protein synthesis by the addition of chloramphenicol. Cells were transferred to fresh media, incubated at 20° C. and resolubilisation of targets had been analysed at different time points. In fact, in the case of Btke the chloramphenicol-induced block of protein synthesis induced a low increment of the soluble recombinant protein in control cells but an impressive increase when specific chaperone combinations were co-expressed simultaneously with the target gene prior to the translational arrest (FIG. 3A). It is worthy to note that for Btke the optimal chaperone combination differed when the soluble protein accumulated during standard culture conditions and when protein synthesis has been blocked (FIGS. 2 and 3A). The choice of time and temperature conditions during re-folding was crucial for optimising the result (FIG. 3B). Longer incubation times or higher temperature lowered the amount of recovered soluble protein, probably because degradation by proteases takes over re-folding activity. As can been seen in Table 1 above, this method of combining chaperone co-overexpression with the blocking of protein synthesis resulted in a great improvement in the yield of recombinant protein in a large number of the combinations tested.

### Example 8

### Testing the Effect of Co-overexpression of Chaperone Combinations and Target Proteins on Re-folding of Aggregated Proteins by Reducing Construct Gene Transcription

The protocol used to block protein synthesis, as described in Example 6 above, was evaluated by means of experiment. It was found that the original protocol can be simplified and that it was not strictly necessary to completely prevent protein synthesis in order to induce re-folding, and in fact the cessation of recombinant protein expression by removing the induction agent (IPTG) was sufficient. In this case the target protein could be re-folded to a level comparable to that obtained in the presence of chloramphenicol but only in the presence of the recombinant over-expressed chaperones (FIG. 3C). For Btke the optimal re-folding conditions enabled the recovery of 42-fold more protein than in the standard growth conditions using normal BL21 (DE3) cells and the simplified protocol (without chloramphenicol) gave an increase factor of 26. We also tried to induce the inclusion body formation culturing the bacteria at 42° C. and starting the re-folding from a higher amount of material but the improvement was negligible (FIG. 3D), probably indicating that the limiting factor is represented from the folding machinery or from the cellular degrading metabolism. These two factors seem to be somehow connected, as illustrated in the case of Tep4. In contrast to Btke this protein was expressed in soluble form at sufficient levels also at standard culture conditions and chaperone co-expression induced a limited yield increase (FIG. 3E). Nevertheless, the suppression of IPTG induction by simple exchange with fresh medium boosted the accumulation of soluble protein in both the strains but only the co-expression of recombinant chaperones could ensure the same results when chloramphenicol was added. Generally, we observed that the addition of fresh medium alone was more effective than the combination of fresh medium and chloramphenicol in strains with wild type chaperone expression. This indicates that the removal of the inducer IPTG, and the subsequent cessation of transcription of the target gene, is sufficient to allow refolding from inclusion bodies. It was a goal of the inventors to obtain more information about the relationship between protein re-folding and degradation by transforming our vectors in the protease deficient strain BB7333. However, the inventors were not able to raise a sufficient number of bacterial colonies. This finding confirmed the general role of proteases in maintaining cell viability (Tomoyasu, T., Mogk, A., Langen, H., Goloubinoff, P., Buckau, B., Mol. Microbiol., 40, 397-413, (2001)) and suggests that a certain degree of protein degradation must be maintained. It is therefore clear from the above example that a reduction in recombinant target gene transcription can also allow the refolding of aggregated proteins to proceed, leading to greatly improved yields of the soluble recombinant protein of interest.

Protein synthesis inhibitors other than chloramphenicol, such as tetracycline, gentamycin and streptomycin have been tested with similar effects.

### Example 9

### The Effect of Co-overexpression of Chaperone Combinations and Target Proteins on Re-folding of Aggregated Proteins in vitro

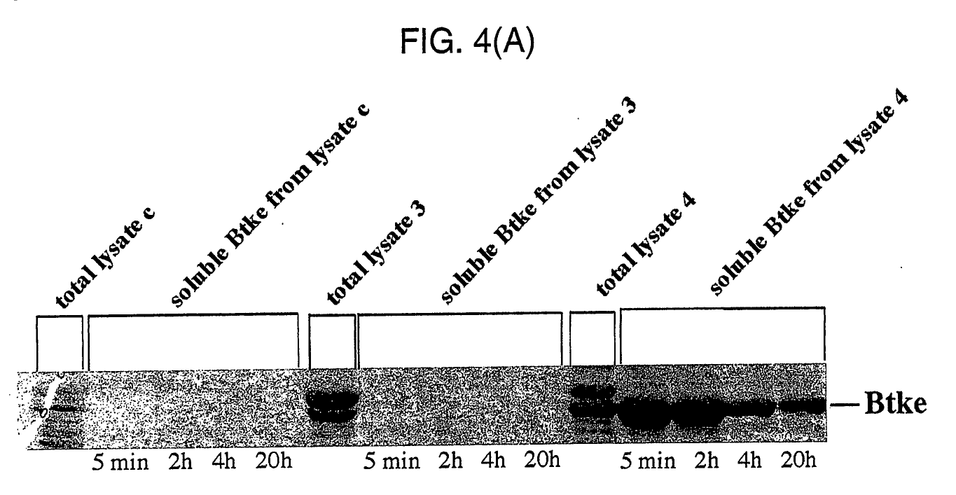

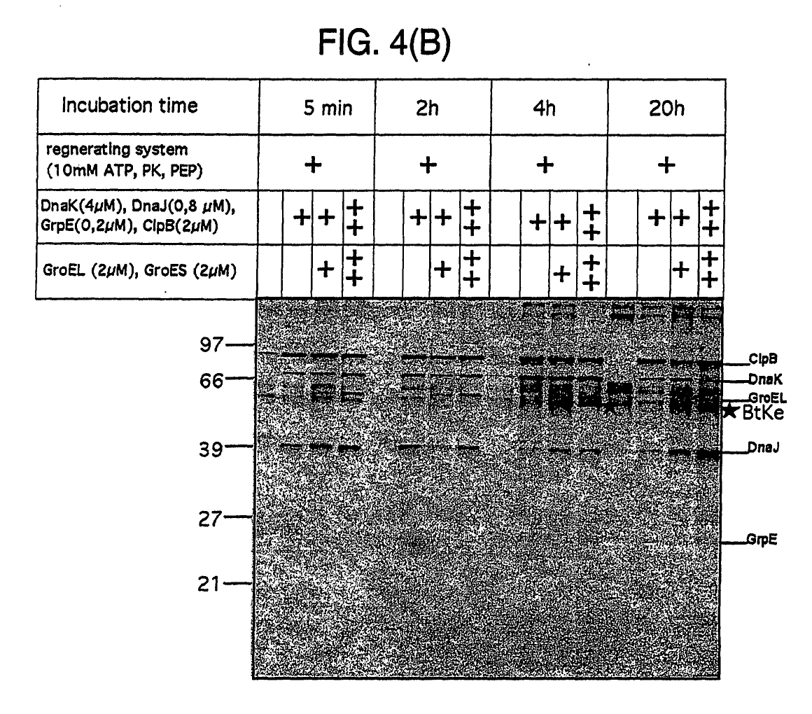

Next, we analysed whether co-expressed chaperones are capable of enhancing the refolding of target proteins from inclusion bodies in vitro after cell lysis. For that purpose, we induced simultaneously synthesis of Btke together with either chaperone combination 3 or 4. Cells were harvested after induction and total lysates containing inclusion bodies and chaperones were isolated. Subsequently an ATP-regenerating system was added to the lysates and the soluble protein was purified after 5 min, 2 h, 4 h and 20 h. Lysate containing the chaperone mixture 4, which was the most efficient during the in vivo refolding of Btke, showed already 5 min after the addition of ATP that approximately all Btke could be recovered in the soluble fraction. The control lysate, where only Btke was overexpressed, and the lysate with enhanced levels of GroEL/EL showed no significant recovery of soluble Btke (FIG. 4A). It is therefore clear that co-overexpressed chaperone mixtures stimulate re-solubilisation of inclusion bodies from bacterial cell lysates. Refolding of Btke inclusion bodies was also possible when chaperones were added exogenously to isolated aggregates (FIG. 4B). However, refolding efficiency was much lower and refolding kinetics much slower, most probably due to the limited amount of added chaperones. This example clearly shows that co-expression of chaperones can also increase the yield of soluble recombinant protein via an enhancement of the refolding of target proteins from aggregates/inclusion bodies in vitro.

The above Examples 1-8 have clearly shown the value of the methods provided by the present invention for increasing protein yield. The re-folding protocol applied to the chaperone transformed cells allowed even higher yields of soluble protein than the simple co-expression with the target proteins in 8 on 17 cases and, importantly, also gave positive results also in the case of 8 constructs insensitive to simple co-expression. Taking all the results together chaperones had a positive effect on soluble protein accumulation in 68% of the cases analysed in our survey. The ratio remains basically the same if all the 50 constructs are considered (34 positive) or if only the 37 different proteins are taken in account (24 positive, 65%). It must be remarked that such a positive result has been obtained despite the fact that most of the constructs used in the experiment correspond to sequences difficult to be expressed in a soluble form in bacteria, like membrane-associated or secreted proteins, regions not corresponding to structural domains or complexes (underlined in Table 1). The advantage of the in vivo disaggregation is that protein refolding follows native patterns and, therefore, recovers its native conformation. The correct folding of some of the proteins was analysed by purification until homogeneity followed by circular dichroism analysis, indicating that the proteins had adopted their native conformation after refolding. Importantly, the enzymatic activities of the kinases B1R and F10L, the TEV protease and luciferase were also recovered after re-folding (data not shown). Larger scale cultures confirmed the trend observed in test cultures, suggesting that the disclosed methods are suitable for industrial applications. In summary, the invention provides not only a method for the production of large amounts of soluble recombinant protein, but also a method for the production of large amounts of recombinant protein that is correctly folded and furthermore retains the native protein's biological activity.

In the following examples 10 and 11, the effect of small heat shock proteins (sHSPs) on the yield of soluble recombinant proteins both in vitro and in vivo was investigated. Published data had previously shown that members of the chaperone family of small heat shock proteins (sHSPs), such as the E. coli family members IbpA and IbpB (IbpAB), can efficiently prevent the aggregation of unfolded proteins, although they were not shown to exhibit protein refolding activity. In the present study, refolding of substrates from sHSP/substrate complexes is reported to be dependent on an Hsp70 chaperone system (such as DnaK with its DnaJ and GrpE co-chaperones) in a reaction that can be further stimulated by the GroEL and GroES (GroELS) chaperones.

### Example 10

### Investigation of the Effect of Small Heat Shock Proteins on the Yield of Soluble Recombinant Proteins in vitro

The refolding of several recombinant proteins from soluble complexes was tested:

Materials and Methods:

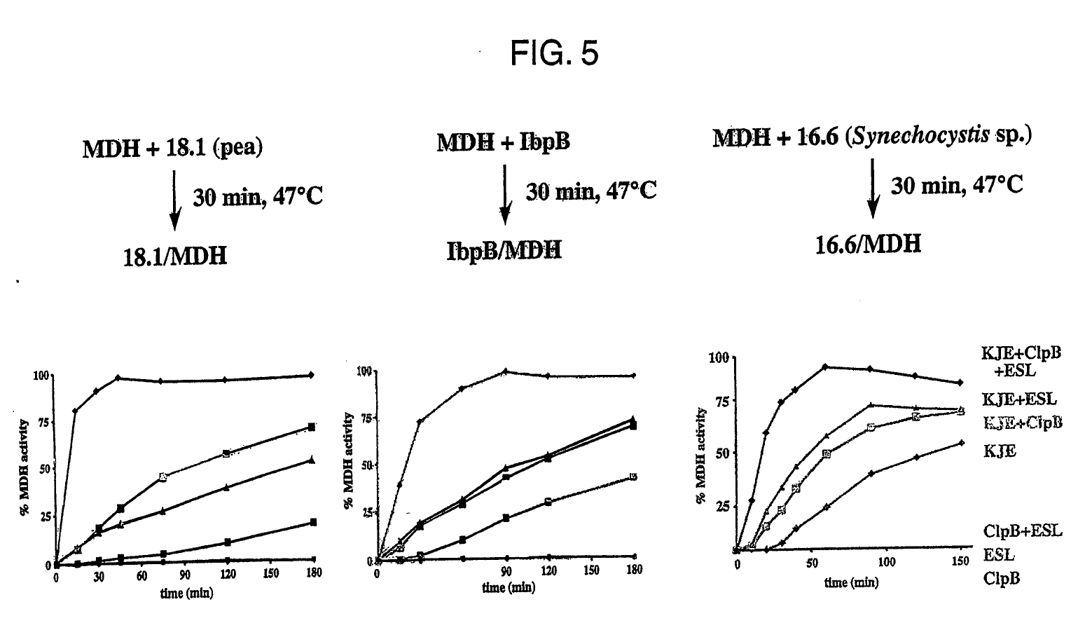

1 μM MDH was denatured in buffer A (50 mM Tris pH 7.5; 150 mM KCl; 20 mM MgCl₂) for 30 min at 47° C. either in the presence of 6 μM 18.1 (pea), or 6 μM IbpB (E. coli), or 4 μM 16.6 (Synechocystis sp.). MDH refolding was initiated at 30° C. by adding an ATP regenerating system (2 mM ATP; 3 mM PEP; 20 ng/ml pyruvate kinase) and various chaperone combinations made up from KJE (1 μM DnaK; 0.2 μM DnaJ; 0.1 ,μM GrpE), ESL (4 μM GroEL; 4 μM GroES) and ClpB (1.5 μM). The results for these experiments are shown in FIG. 5.

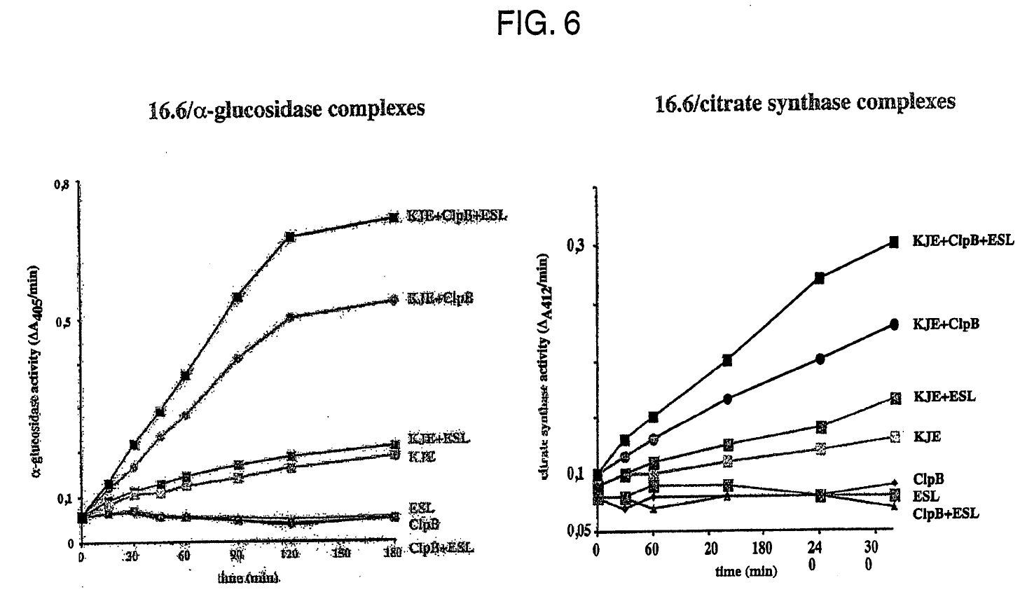

Similarly, 1 μM α-glucosidase or 1 μM citrate synthase were denatured in the presence of 4 μM 16.6 (Synechocystis sp.) in buffer A for 45 min at 50° C. or 47° C., respectively. Protein refolding was initiated at 30° C. by adding an ATP regenerating system (2 mM ATP; 3 mM PEP; 20 ng/ml pyruvate kinase) and various chaperone combinations made up from KJE, ESL and ClpB. The results for these experiments are shown in FIG. 6.

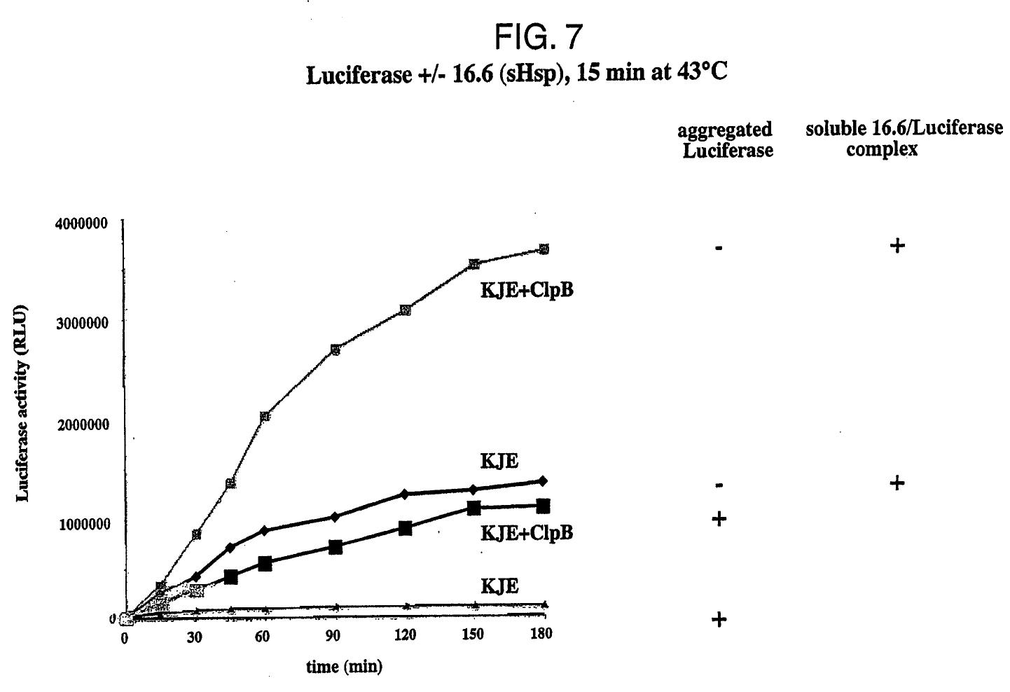

Similarly, 100 nM firefly luciferase was denatured in the absence or presence of 0,4 μM 16.6 (Synechocystis sp.) in buffer A for 15 min at 43° C. Luciferase refolding was initiated at 30° C. by adding an ATP regenerating system (2 mM ATP, 3 mM PEP; 20 ng/ml pyruvate kinase) and various chaperone combinations made up from KJE (0.5 μM DnaK; 0.1 μM DnaJ; 0.05 μM GrpE) and ClpB (0.5 μM). The results for these experiments are shown in FIG. 7.

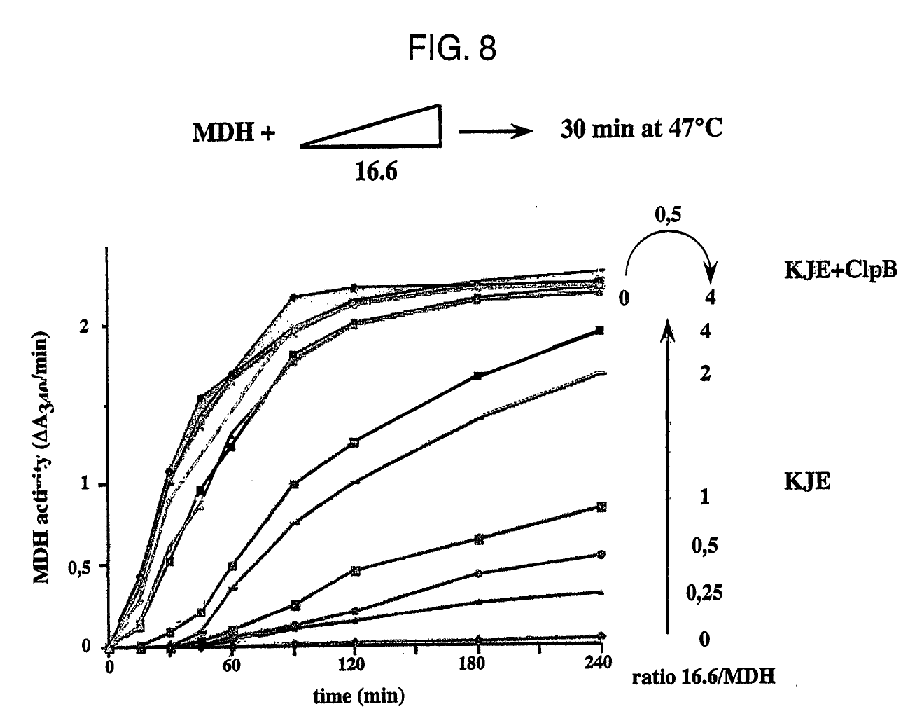

To investigate the effect of the stoichiometry of the sHSPs on the refolding of sHSP/substrate complexes 1 μM MDH was denatured in buffer A (50 mM Tris pH 7,5; 150 mM KCl; 20 mM MgCl₂) for 30 min at 47° C. in the presence of varying 16.6 concentrations. MDH refolding was initiated at 30° C. by adding an ATP regenerating system (2 mM ATP; 3 mM PEP; 20 ng/ml pyruvate kinase) and various chaperone combinations made up from KJE (1 μM DnaK; 0.2 μM DnaJ; 0.1 μM GrpE) and ClpB (1.5 μM). The results for these experiments are shown in FIG. 8.

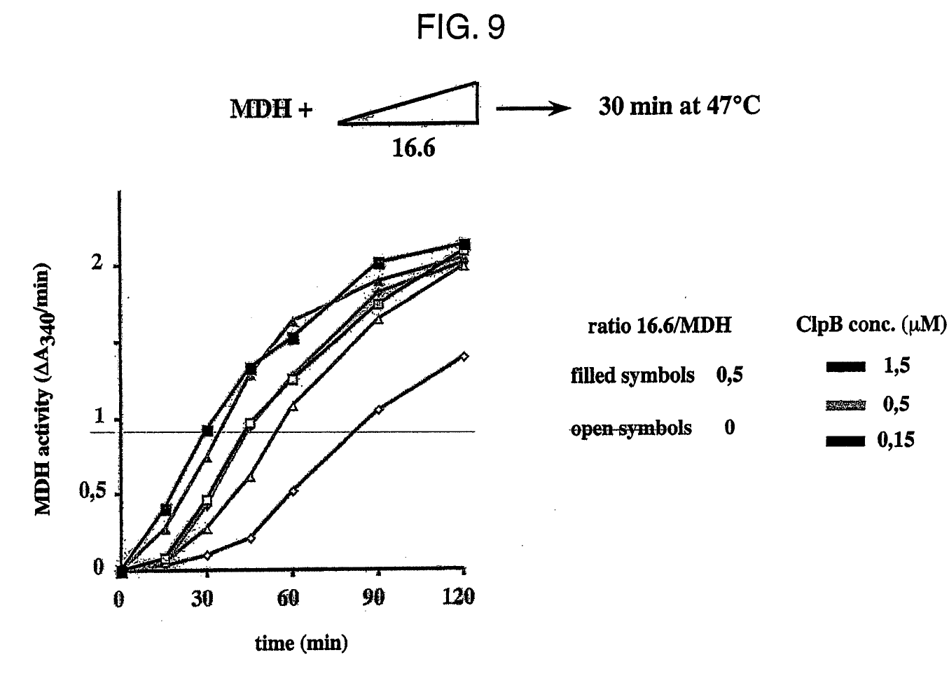

Experiments were also carried out in which 1 μM MDH was denatured in buffer A (50 mM Tris pH 7.5; 150 mM KCl; 20 mM MgCl₂) for 30 min at 47° C. in the absence or presence of 0.5 μM 16.6. MDH refolding was initiated at 30° C. by adding an ATP regenerating system (2 mM ATP; 3 mM PEP; 20 ng/ml pyruvate kinase) and the DnaK system (1 μM DnaK; 0.2 μM DnaJ; 0.1 μM GrpE) in the presence of varying ClpB concentrations as indicated. The results for these experiments are shown in FIG. 9.

Results:

All the sHSPs tested formed complexes with heat-denatured protein substrates such as malate dehydrogenase (MDH), firefly luciferase and alpha-glucosidase which represented small protein aggregates. The data shown in FIG. 5 show that ClpB strongly stimulates the DnaK-dependent refolding of the thermolabile reporter protein malate dehydrogenase (MDH) from various soluble sHSP/MDH complexes. This stimulatory effect was verified by analysis of the refolding of the substrates firefly luciferase, citrate synthase and α-glucosidase from complexes with sHSP 16.6 (shown in FIG. 6 and FIG. 7). Notably, the refolding of substrates by ClpB/DnaK from sHSP/substrate complexes was in general much faster than refolding from aggregated proteins generated by identical denaturation conditions in the absence of sHSPs (FIG. 7). The GroESL chaperone system was not able to refold any of the substrates tested from sHSP/substrate complexes, even in the presence of ClpB. However GroESL was observed to increase the rates of substrate refolding in the presence of DnaK or ClpB/DnaK, especially in case of MDH (FIG. 5). Table 2 provides a summary of the results from these experiments:

TABLE 2
Refolding of thermolabile proteins from protein aggregates or
soluble sHsp/protein complexes

| | Chaperones | | | |
|---|---|---|---|---|
| Substrate | KJE | KJE/ESL | KJE/ClpB | KJE/ClpB/ESL |
| aggr. MDH | 0.1 | 0.2 | 10.3 | 25.1 |
| sHsp/MDH | 4.0 | 9.9 | 8.5 | 27.5 |
| aggr. α-glucosidase | 0 | 0 | 1.73 | 2.27 |
| sHsp/α-glucosidase | 0.44 | 0.53 | 2.69 | 3.63 |
| aggr. citrate synthase | 0 | 0 | 0.06 | 0.1 |
| sHsp/citrate synthase | 0.12 | 0.22 | 0.4 | 0.63 |
| aggr. luciferase | 0.01 | n.d. | 0.14 | n.d. |
| sHsp/luciferase | 0.17 | n.d. | 0.48 | n.d. |

Refolding rate (nM/min)
MDH, α-glucosidase, citrate synthase and luciferase were denatured in the absence or presence of a 4-fold excess of 16.6. Substrate refolding was initiated by addition of an
# ATP-regenerating system and the indicated chaperone combinations (experimental details as described above). Maximal rates of substrate refolding were derived from the linear
# phase of the time curves of recovered enzymatic activity.

On the basis of these results, we propose that sHSP/substrate complexes represent small protein aggregates and refolding of substrates from such complexes relies on a disaggregation reaction mediated by the DnaK system alone, or much more efficiently by ClpB with the DnaK system. After their active extraction from the complex, unfolded substrates are subsequently refolded by a chaperone network formed by the DnaK and GroESL systems.

In vivo the levels of sHSPs are often not sufficient to prevent protein aggregation and sHSPs are usually found associated with protein aggregates. We investigated whether the presence of sHSPs in protein aggregates can facilitate their resolubilization and consequently increase substrate refolding. To answer this question the amount of sHSPs utilised in each experiment was titrated during the denaturation of MDH and the resulting consequences on DnaK or DnaK/ClpB-mediated MDH refolding were investigated. Substiochiometric concentrations of Hsp16.6 compared to MDH resulted in the formation of insoluble, turbid sHSP/MDH complexes which were, however, much smaller than MDH aggregates formed by denaturation in the absence of Hsp16.6 (Table 3).

TABLE 3
Characterisation of 16.6/MDH complexes

| | | | Size determination | |
|---|---|---|---|---|
| | | | Dynamic | |
| 16.6/ | | | lightscattering | Static |
| MDH | Lightscattering | Solubility | Calculated | lightscattering |
| Ratio | intensity (%) | (%) | radius (nm) | Mass (Da) |
| 0 | 100 | <10 | 45 +/− 15 | n.d. |
| 0.25 | 68 | <10 | 33.7 +/− 12.5 | 1.8E+07-7.0E07 |
| 0.5 | 37 | 18 | 31.5 +/− 9 | 1.8E+07-7.0E+07 |
| 1 | 0 | 57 | 24 +/− 6 | 5.6E+07-1.5E+07 |
| 2 | 0 | 84 | 19 +/− 5 | 2.3E+06-4.0E+06 |
| 4 | 0 | 92 | 14 +/− 5 | 1.5E+06-3.1E+06 |
| 1 μM MDH was denatured in buffer A (50 mM Tris pH 7.5; 150 mM KCl; 20 mM MgCl₂) for 30 min at 47° C. in the presence of varying 16.6 concentrations, | | | | |
| # given as 16.6/MDH ratio. Turbidity (light scattering intensity) of formed MDH aggregates was set at 100%. Solubility of native, untreated MDH after centrifugation | | | | |
| # (13.000 rpm, 15 min, 4° C.) was set 100%. Size of the different sHSP/substrate complexes were determined either by dynamic or static lightscattering | | | | |
| # (coupled to gelfiltation) measurements. Both techniques were utilised in case of poorly soluble sHSP/MDH complexes leading to characterization of a subpopulation of the complexes only. | | | | |

Increasing Hsp16.6 concentrations increased the solubility and decreased turbidity and size of sHSP/MDH complexes (Table 3). Efficient DnaK-dependent MDH refolding required the presence of soluble sHSP/MDH complexes created in the presence of high Hsp16.6 concentrations (FIG. 8). In contrast ClpB/DnaK mediated MDH refolding did not show up such a severe dependency, however MDH activity was recovered at earlier timepoints if insoluble sHSP/MDH complexes instead of MDH aggregates were used as starting material. This effect became much more severe, if the disaggregation potential of the ClpB/DnaK system was reduced by lowering the ClpB concentration (FIG. 9). The stimulatory effects described above were again observed when substoichiometric concentrations of sHSPs were present during substrate denaturation (by heat), resulting in the formation of insoluble sHSP/substrate complexes. Thus the presence of sHSPs in insoluble protein aggregates can significantly facilitate aggregate resolublization by ClpB/DnaK.

The above example illustrates that refolding of substrates after their ClpB/DnaK mediated extraction from sHSP/substrate complexes is in most cases stimulated by the GroESL chaperone system, indicating that released, unfolded substrates are refolded by a chaperone network. We conclude that sHSP function is coupled to ClpB/DnaK dependent protein disaggregation and serves to prepare protein aggregates for faster resolubilization.

### Example 11

### Investigation of the Effect of Small Heat Shock Proteins on the Yield of Soluble Recombinant Proteins in vivo

Materials & Methods:

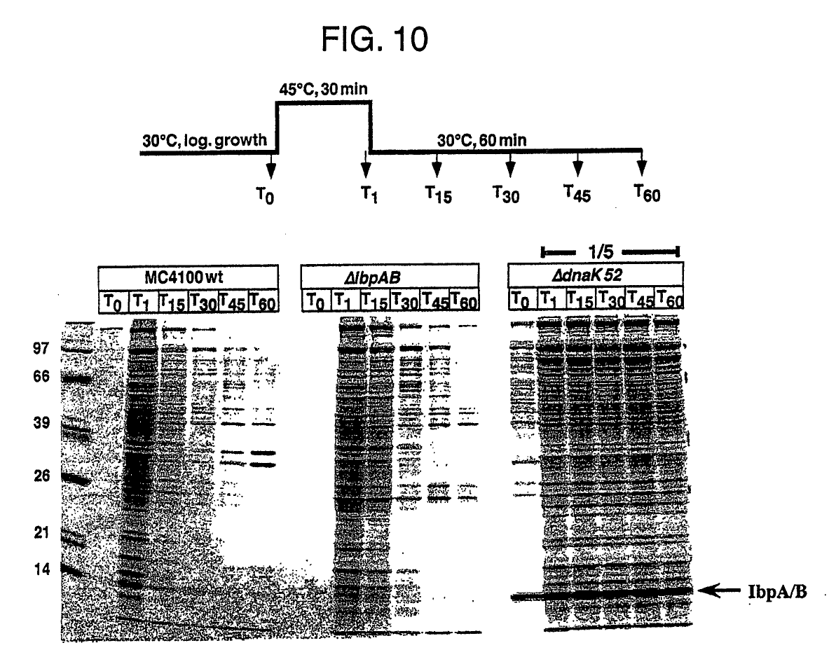

E. coli wild type or ΔibpAB or ΔdnaK mutant cells were grown at 30° C. to logarithmic phase and shifted to 45° C. for 30 min, followed by a recovery phase at 30° C. for 60 min. Protein aggregates were isolated at the indicated timepoints and analyzed by SDS-PAGE. The results for these experiments are shown in FIG. 10.

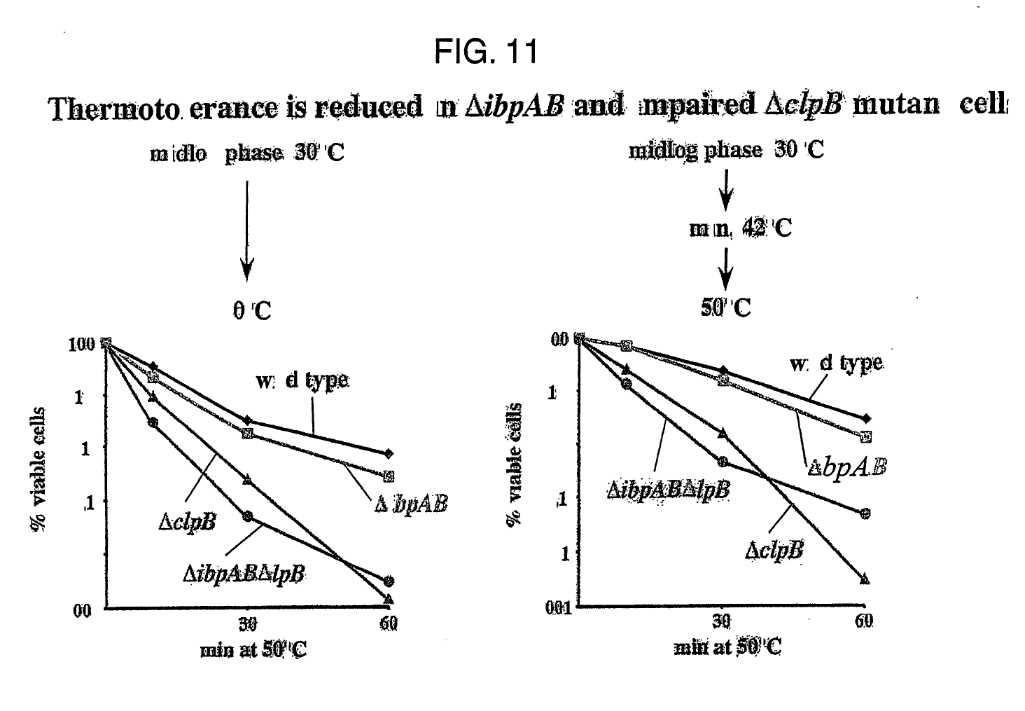

E. coli wild type or ΔibpAB or ΔclpB or ΔibpAB ΔclpB double mutant strains were grown at 30° C. to logarithmic phase. Cells were either shifted directly to 50° C. or were preincubated at 42C. for 15 min. Various dilutions of stressed cells were plated on LB plates. After 18 h colony numbers were counted and survival rates were calculated in relation to determined cell numbers before 50° C. shock. The results for these experiments are shown in FIG. 11.

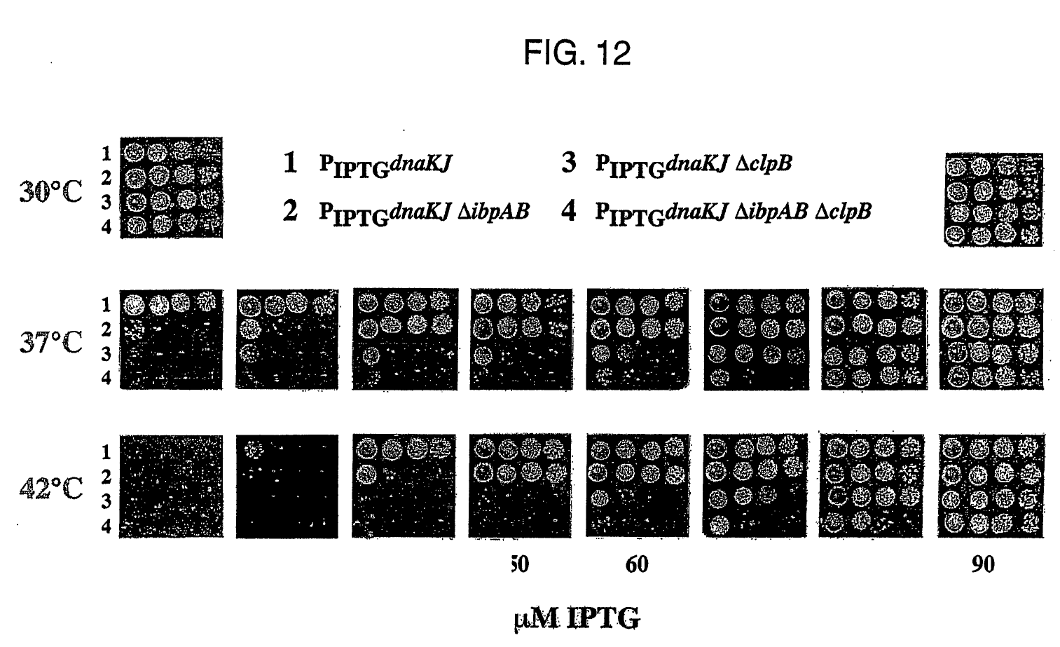

Various dilutions (10⁻³ to 10⁻⁶) of the cultures were spotted on LB plates supplemented with the indicated IPTG concentrations and incubated at 30° C., 37° C. or 42° C. for 18 h. The results for these experiments are shown in FIG. 12.

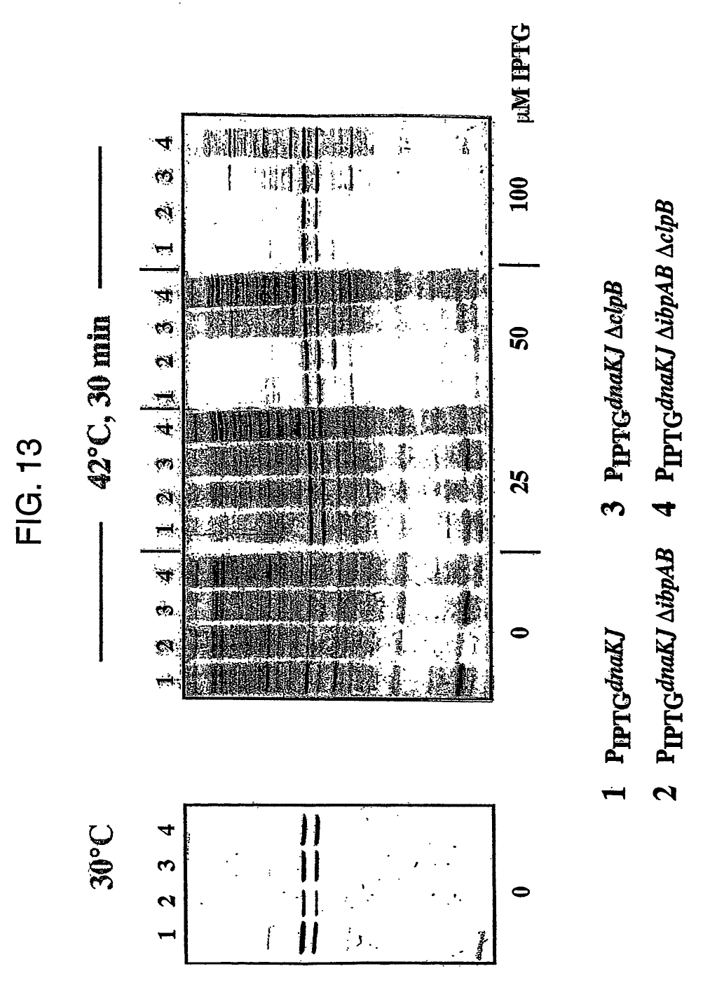

Various strains of E. coli were grown overnight at 30° C. in the presence of 500 μM IPTG. Cultures were washed twice with LB and inoculated for further growth at 30° C. in the presence of various IPTG-concentrations (0, 25, 50, 100 μM) to logarithmic phase and shifted to 42° C. for 30 min. Protein aggregates were isolated at the indicated timepoints and analyzed by SDS-PAGE. The results for these experiments are shown in FIG. 13.

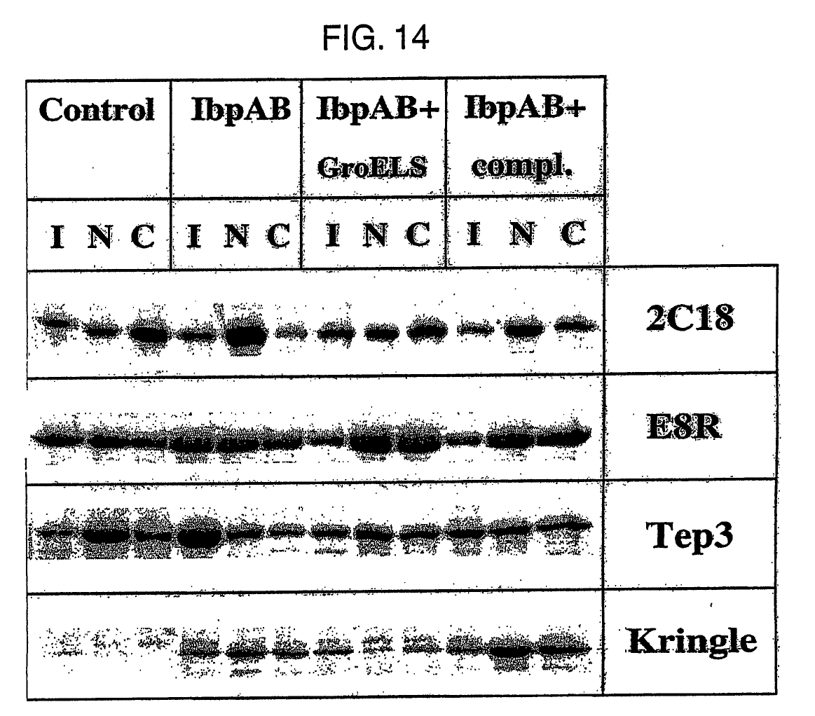

In the experiments described above in examples 6-9 we expressed in E. coli strain BL21 (DE3) several target proteins including 2C18, E8R, Tep3 and Kringle with or without co-expression of different combinations of the chaperones GroELS, ClpB, DnaK, DnaJ and GrpE. The chaperone combination which for each case yielded the highest levels of soluble target proteins was taken as “control” (overproduction of KJE/ELS/B for 2C18, Tep3, no chaperone overproduction for E8R and Kringle). To show the solubilization effects of overproduction of IbpA/IbpB together with other chaperones we generated BL21(DE3) strains which carry plasmids expressing IPTG-regulatable genes encoding these same target proteins and in addition plasmids expressing IPTG regulatable genes encoding IbpA/IbpB (lanes marked IbpAB in FIG. 14), IbpA/IbpB and GroELS (lanes marked IbpAB+GroELS in FIG. 14), IbpA/IbpB and GroELS and DnaK/DnaJ/GrpE and ClpB (lanes marked IbpAB+compl. in FIG. 14). After IPTG induction the bacteria were cultured overnight at 20° C. and directly collected (I), or the IPTG was removed and the pellet re-suspended in fresh medium and cultured for two additional hours without (N) or with 200 μg/ml of chloramphenicol (C). For each combination the amount of soluble protein (after affinity purification of the target proteins in the soluble cell fractions) was identified on Coomassie-stained SDS-gels. The results for these experiments are shown in FIG. 14.

Results:

E. coli mutant cells missing the sHSPs IbpA/B do not exhibit a temperature-dependent growth phenotpye (42° C.). However, we observed that the resolubilization of protein aggregates, created by severe heat treatment (45° C.), was delayed in comparison to wild type cells (FIG. 10). Additionally the survival rate (thermotolerance) of ΔibpAB mutants at lethal temperatures (50° C.) was slightly reduced compared to wild type (FIG. 11). Thermotolerance is linked to the ability of cells to rescue aggregated proteins and consequently the observed reduced thermotolerance of ΔibpAB mutants is likely caused by a less efficient resolubilization of protein aggregates.

DnaK has been shown to be the major player in preventing protein aggregation in E. coli at high temperatures. We therefore investigated whether IbpA/B function could become more important in the presence of reduced DnaK levels, rendering E. coli cells more sensitive to protein aggregation. In vivo depletion of DnaK was achieved by replacing the σ32-dependent promotor of the dnaKJ operon by an IPTG-inducible one. Reduced DnaK levels caused synthetic lethality in ΔibpAB mutant cells at elevated temperatures (37-42° C.). The same experiments performed in a ΔclpB mutant strain and a ΔibpAB ΔclpB double knockout revealed an increasing necessity for higher DnaK levels at elevated temperatures (FIG. 12). Especially in case of the ΔibpAB ΔclpB double knockout mutant strain this phenotype was linked to severe protein aggregation upon heat shock to 42° C. (FIG. 13). Thus in vivo IbpA/B is necessary for efficient protein disaggregation, especially under conditions which favour protein aggregation and lower the disaggregation potential of cells.

As shown in FIG. 14 and Table 4, the combined overproduction of IbpAB with ClpB, the DnaK system and the GroEL system, and with combinations of these chaperones, increases the yield of soluble recombinant protein produced in E. coli cells.

TABLE 4

| Protein | MW | Organism | Features | IpbAB IF |
|---|---|---|---|---|
| SerprotAg1 | | A. gambiae | domain/fus | ! |
| Kringle | 30 kD | H. sapiens | domain/fus | ! |
| 2C18 | 50 kD | H. sapiens | Fl/fus | 2.5 |
| 22j21 | 72 kD | H. sapiens | Fl/fus | 0 |
| Tep3 | 70 kD | A. gambiae | domain/fus/secr | 3.5 |
| Tep4 | 68 kD | A. gambiae | domain/fus/secr | 0 |
| XklpA3 | 73 kD | X. laevis | domain/fus/cyt | 0 |
| E8R1 | 58 kD | Vaccinia virus | Fl/membr/fus | 3.5 |
| BtKe | 55 kD | H. sapiens | domain/cyt | 0 |
| Nine proteins were tested for the effects of IpbAB co-expression on the level of soluble target proteins produced in E. coli cells. The increment factor (IF) defines the fold increase (in the best condition, being either I, N or C; | | | | |
| # see above for definition) in amount of soluble protein due to IpbAB co-expression with respect to the controls (the best conditions identified from examples 6-9). | | | | |
| # ! denotes that the IpbAB-dependent expression of soluble proteins occurred which could not be produced in soluble form before. Thus, in 5 of the nine cases tested, the overproduction of IbpA/IbpB further increasesd the yield of target proteins. | | | | |

Thus, these in vivo data are consistent with the results obtained in vitro. Firstly, the yields of soluble recombinant protein produced in E. coli cells can be increased in several cases tested when IbpA/IbpB is overproduced alone or together with various combinations of the DnaK and GroELS systems and ClpB. Secondly, E. coli ΔibpAB mutant cells missing IbpA/B exhibited a delayed protein disaggregation after heat shock (45° C.) and a reduced survival rate at lethal temperatures (50° C.) compared to wild type cells. IbpA/B function became essential at elevated temperatures (37-42° C.) in the presence of reduced DnaK levels, conditions which favour protein aggregation and reduce the disaggregation potential of the cells.

In summary, the above Examples 10 and 11 show that small heat shock proteins (sHSPs) co-operate with other chaperones, in particular with the ClpB chaperone, the DnaK chaperone system and the GroEL chaperone system, to solubilize and refold aggregation-prone proteins. This property can be exploited to increase the yield of soluble recombinant proteins produced in E. coli and other cells, and can be used for the in vitro production of soluble recombinant protein. In particular, the combined overproduction of IbpAB with ClpB, the DnaK system and the GroEL system, and with combinations of these chaperones, increases the yield of soluble recombinant protein produced in E. coli cells. However, the teaching provided by these experiments is of much broader importance since all the proteins involved in this folding reaction are members of large protein families with members among prokaryotes and eukaryotes (IbpA and IbpB are members of the family of sHSPs which includes alpha-cristallins; ClpB is member of the AAA protein family which include Hsp104; DnaK is member of the Hsp70 family; DnaJ is member of the DnaJ (Hsp4O) family; GrpE is member of the GrpE family; GroEL is member of the Hsp60 family; GroES is member of the GroES family). It is expected that the other members of the involved protein families can substitute for the E. coli members in protein folding reactions. In fact, we present biochemical data that the sHSP of Synechocystis, Hsp16.6, can increase the efficiency of protein refolding in cooperation with the E. coli chaperones ClpB, DnaK, DnaJ, GrpE, and GroELS. Furthermore, since ClpB is a homolog of the S. cerevisiae Hsp104, a chaperone implicated in the generation and prevention of formation of amyloid fiber formation, it is also possible that our finding that the sHSPs co-operate with ClpB and the DnaK and GroEL systems in protein folding has implications on the formation or treatment of amyloid fibers in eukaryotic cells, and diseases in which such fibers are implicated.

Finally, it was found that the IbpA/B, ClpB and the DnaK systems act cooperatively to reverse protein aggregation.

To elucidate the functional interplay between IbpA/B, KJE and ClpB in the protein quality control network more precisely, we determined the degree of protein aggregation in ΔibpAB, ΔclpB and ΔibpAB ΔclpB mutants that have KJ adjusted to various levels. Since ClpB and IbpA/B do not prevent protein aggregation in vivo (Mogk et al., 1999), increased amounts of aggregated proteins in the respective mutants would indicate a less efficient protein disaggregation. At 30° C. no protein aggregation was detectable for all tested mutant strains, even in cells with greatly reduced KJ levels. After a 30 min incubation at 42° C., 5% of cellular proteins aggregated in all mutant cells, provided that IPTG was omitted from the growth medium. Increasing KJ levels (by addition of IPTG) reduced the amount of aggregated proteins in each strain, but to different degrees dependent on the mutant background. While 50 μM IPTG in the growth medium was sufficient to eliminate aggregates in ΔibpAB cells, 2% and 5% of total proteins still aggregated in ΔclpB and ΔibpAB ΔclpB mutant cells, respectively. Even in presence of DnaK/DnaJ levels corresponding to heat shock conditions (100 μM IPTG), 2% of cellular proteins remained aggregated in ΔibpAB ΔclpB mutant cells.

These findings are in complete agreement with the hierarchial complementation of growth defects of these mutant cells at high temperatures and demonstrate the cooperative action of IbpA/B and ClpB in the KJE-mediated removal of protein aggregates in vivo.

### Example 12

### Bacteria Co-Transformed with Recombinant Proteins and Chaperones Cloned in Independent Plasmids are Suitable for Expression Tuning

This example describes a system based on three vectors, where two are under IPTG regulation and enable the recombinant expression of six chaperones, and the third one is arabinose-inducible and harbours the sequence for the recombinant target protein of interest. In such a way, the independent induction and the level of expression of both chaperones and target protein was possible. The data showed that the expression leakage from pET vectors was prevented by the introduction of further plasmids in the cell and that the recombinant proteins compete for their expression. In fact, the high rate induction of one of them could switch off the accumulation of the other recombinant proteins. The first information was used to maximise the expression of toxic proteins while the cross-inhibition among recombinant proteins was exploited to modulate and optimise the target protein expression and to induce the chaperone-assisted in vivo re-folding of aggregated target protein.

### Cloning and Transformation Procedures.

Chaperone proteins were expressed as described above. For expression of target protein, the sequences corresponding to GTR1 (O 00582) and the motor regions of Xklp3A and Xklp3B (AJ 311602; CAA 08879) were cloned in pTrcHis vector (trc promoter and ColE1 replication origin), Tep3 (unpublished sequence from A. gambiae) was cloned in pGEX (tac promoter and pBR322 replication origin) and E8R (NP 063710) in pGAT (lac promoter and pUC replication origin). The sequences for GTR1, Tep3, E8R, the Xklp3A and B C-terminal regions of the coil-coiled domains and a domain of Btk (O 06187) were cloned in pBAD. pET24d and pETM60. The Xklp3A and B C-terminal regions of the coil-coiled domains were also cloned in the polycistronic vector pST39 (Tan, 2001).

Cell Cultures.

Single colonies from the transformed cells were used to inject 3 mL of LB medium. Liquid cultures were incubated initially at 37° C., successively transferred to 30° C. or 20° C., induced at an OD₆₀₀ of 0.8 and grown 3 hours or overnight, respectively. Variations of timing and concentration combinations used in the experiments with bacteria hosting both IPTG and arabinose regulated expression vectors are described case by case in the results.

Protein Purification and Yield Evaluation.

Frozen bacterial pellets corresponding to 0.5 mL of culture were re-suspended in 350 μL of 20 mM Tris HCl, pH 8.0, 2mM PMSF, 0.05% Triton X-100 and 1 mg/mL lisozyme and incubated on ice for 30 min, with periodic stirring. The suspension was sonicated in water for 5 minutes, pelleted in a minifuge, the supernatant was added to 20 μL of pre-washed Ni-NTA magnetic agarose beads (Qiagen) and incubated further 30 min under agitation before being removed. Beads were washed 30 min with 20 mM K-phosphate buffer, pH 7.8, 300 mM NaCl, 20 mM imidazole, 8% glycerol, 0.2% Triton X-100 and later with PBS buffer plus 0.05% Triton X-100. Finally they were boiled in 12 μL SDS sample buffer and the samples loaded onto a SDS PAGE using a Pharmacia rninigel system. Proteins were detected after coloration with Simply Blue Safestain (Invitrogen) following the manufacturer's instructions and the gels were recorded using a Umax Astra 4000U scanner. Proteins were quantified analysing the gel bands with the public domain NIH Image program (developed at the U.S. National Institutes of Health and available on the Internet at http://rsb.info.nih.gov/nih-image/).

Results and Discussion

Initially we transformed bacteria with chaperone-carrying plasmids and in a second step these cells were re-transformed with a plasmid harbouring the target protein. The expression of all the plasmids was under IPTG regulation. The cell co-transformation with three plasmids selected using different antibiotic resistances induced a 20% decrease of the cell growth rate; however, succeeded even in the case in which two plasmids (PGAT and pBAD) shared the same replication origin pUC.

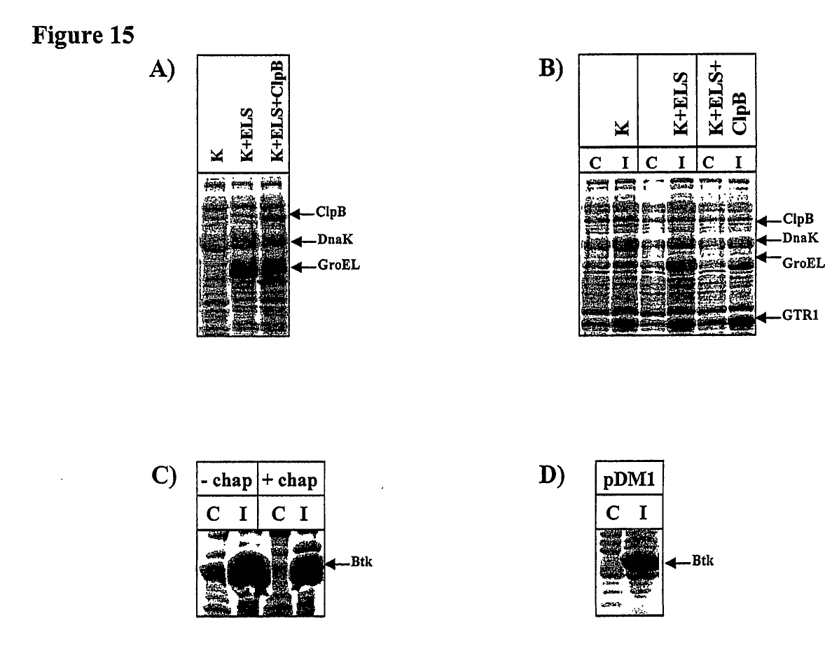

Bacteria transformed with two low copy number plasmids derived from pDM1 and harbouring different chaperone genes expressed the corresponding proteins at very high level (FIG. 15A). Nevertheless, the intensity of the bands separated in SDS-gel indicated 10 that the expression of the target protein GTR1 cloned into the pTrcHis vector strongly inhibited the chaperone accumulation (compare FIGS. 15A and 15B) so that ClpB was no more detectable in the bacterial homogenate (FIG. 15B). In contrast, the expression of the target protein Btk by the leaking vector pET24d in the absence of the inducer IPTG was strongly repressed when a chaperone-containing plasmid was co-transformed in the host cell (FIG. 15C). These results suggest that there are two independent kinds of interaction raising from the presence of different plasmids in the same cell. The first one involves the plasmids and is independent from their protein products. In fact, the IPTG-independent expression of Btk cloned in a pET24d expression vector was prevented also in the case of the co-transformation with an empty pDM1 vector (FIG. 15D). A recent paper reports that the introduction of heterologous vectors has been shown to induce stress responses and inhibit biomass production in S. cerevisiae even though they were empty or non-induced (Görgens et al., 2001, Biotechnol. Bioeng. 73, 238-245).

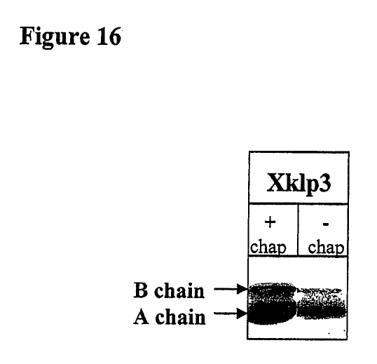

The expression-leakage control obtained by co-transformation with more plasmids at once can be useful in the case of the expression of toxic proteins or when the leakage rate is so high to impair the normal cell function. At least one experience in the frame of this work indirectly supports this hypothesis. A polycistronic plasmid (Tan, 2001) has been used for expressing a complex between the C-terminal end of the coil-coiled regions of Xklp3 chain A and chain B. No colony grew using BL21 (DE3) bacteria when we tried to transform them with the polycistronic plasmid. Cells co-transformed with chaperone plasmids were efficiently transformed with the polycistronic vector and gave colonies. Colonies grew also when the polycistronic vector was transformed into pLysS strain cells and 1% glucose was added to the growth medium to tightly control any expression leakage. However, the bacterial yield was 60% less (data not shown) and the purified protein decreased of more than 80% (FIG. 16).

Beside the case of plasmid interaction our results seem to indicate that the cell machinery involved in the protein production was challenged by the contemporary over-expression of too many recombinant proteins. The results of FIG. 15A and 15B could be interpreted either as an overwhelming accumulation of target protein transcripts that inhibits the chaperone expression rate or a competition for the RNA polymerase. Such a competition has been described in E. coli between metabolic and recombinant genes (Schweder et al., 2002, Appl. Microbiol. Biotechnol. 58, 330-337) while recombinant and cell mRNAs could compete at transcriptional level in yeast (Görgens et al., 2001, Biotechnol. Bioeng. 73, 238-245). We observed that in case of co-transformation the effect of competition seems proportional to the estimated copy number of the target protein plasmid and independent on the promoter used (data not shown). In fact, recombinant vectors hosting the target protein with both T7 and lac promoters could inhibit the chaperone expression. Therefore, a competition at the transcriptional level would be ruled out in our system. The existence of a limit of total protein expression can have important consequences in the case in which recombinant chaperones are co-transformed to boast the production of a target protein. In fact, a too high level of expression of the latter could automatically inhibit the chaperone expression levels and, therefore, limit or prevent their positive folding effect.

An alternative method has been envisaged in which chaperones and target proteins were cloned in vectors in which their expression was under different regulation systems. This enables the independent induction of chaperone and target protein expression and would allow exploitation of the chaperone-dependent folding improvement of the target protein avoiding any shortcomings due to contemporary co-expression. A logical approach seemed to induce the accumulation of the chaperones and then trigger the target protein expression in a cell with boasted folding machinery.

In a first set of experiments the GroELS chaperones were expressed by means of an arabinose-regulated vector (Castanié et al., 1997, Anal. Biochem. 254, 150-152) and the IPTG-dependent target proteins were induced after 30 minutes. The results did not show a significant increase of soluble target proteins and no improvement was detected varying incubation times and inducer concentrations (data not shown).

In a second attempt the target proteins were cloned into arabinose-regulated vectors while five different IPTG-dependent chaperone combinations were compared. Such an expression system mostly resulted in an increased yield of the soluble target proteins (see Table 5).

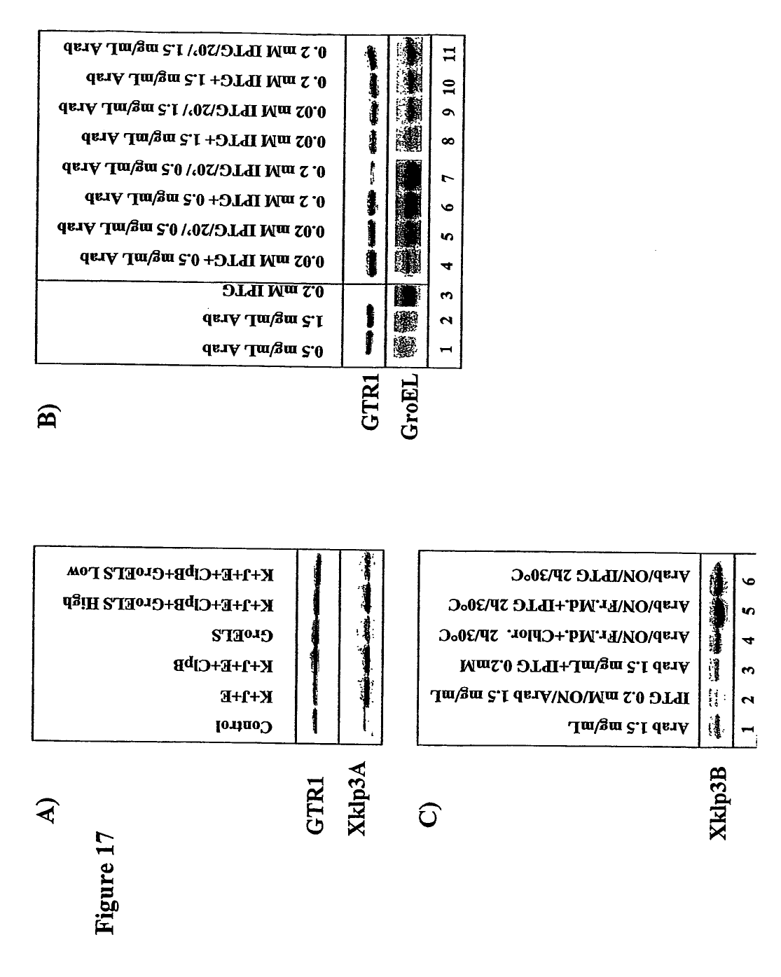

TABLE 5
Chaperone-dependent yield improvement of soluble target proteins.
Clones corresponding to the target proteins were co-transformed with
the different chaperone combinations and cultured according to the
best among the conditions reported in FIG. 17. The improvement factor
enabled by chaperone co-expression indicates the ratio between the
highest yield of soluble target protein obtained using cells
co-transformed with chaperones and its amount recovered from cells
not hosting recombinant chaperones; the symbol ∞ means that no
soluble target protein was expressed in absence of chaperones. The
target proteins were expressed in arabinose-regulated pBAD vectors
and the different chaperone combinations listed in material and
methods were induced by IPTG addition.

| Protein | MW | Organism | Improvement Factor |
|---|---|---|---|
| GTR1 | 40 kD | S. cerevisiae | 3 |
| Btkp | 55 kD | H. sapiens | 3 |
| Xklp3A | 62 kD | X. laevis | ∞ |
| Xklp3B | 40 kD | X. laevis | 9 |
| Tep3 | 70 kD | A. gambiae | 4 |
| E8R | 32 kD | Vaccinia virus | 0 |

The optimal chaperone combination (FIG. 17A) and the expression conditions were specific for each target protein. The complexity of the interactions among the different recombinant proteins is illustrated in the experiments summarised in FIG. 17B and 17C. Soluble GTR1 accumulation was induced at a similar level by both 0.5 and 1.5 mg/mL of arabinose (FIG. 17B, lanes 1 and 2). The co-expression of low amounts of K+J+E+ClpB+GroELS chaperones induced by 0.02 mM IPTG stimulated the accumulation of soluble GTR1 whose expression was induced by 0.5 mg/mL of arabinose (FIG. 17B, lane 4).

Nevertheless, the amount of the soluble target protein decreased if IPTG-dependent chaperones were allowed to accumulate before the arabinose-dependent induction of GTR1 (FIG. 17B, compare lanes 4 and 5). The same pattern of inhibition of soluble GTR1 was observed when higher chaperone expression was induced by ten-fold higher IPTG concentration but, in such a case, the absolute amount of soluble GTR1 was strongly reduced (lanes 6 and 7). These data confirm the existence of a competition among the products of different recombinant plasmids. In this case, both plasmids use the cell RNA polymerase and, therefore, it is not possible to distinguish between competition at the transcription or translation level. Nevertheless, the accumulation of soluble GTR1 induced at low level of arabinose is progressively inhibited by an increasing amount of available chaperones (FIG. 17B). The inhibitory chaperone accumulation was obtained with both higher IPTG concentration and longer time of induction before the arabinose-dependent induction of GTR1. When we repeated the same experiments using 1.5 mg/mL of arabinose to induce a higher GTR1 expression the results were reversed (FIG. 17B, compare lanes 8-11 and 4-7). As a matter of fact the higher arabinose concentration enabled a strong accumulation of GTR1; however, the increasing amounts of expressed chaperones did not reach a level critical for competition but could provide a more stabilising environment for GTR1.

The conclusions from this work are that chaperones can positively contribute to GTR1 accumulation. Nevertheless, a ratio among the transcripts seems to be important for avoiding detrimental competition at the translation level. The parameters involved are the rate of induction of both chaperone and target genes and the time in which chaperones can accumulate before the target protein is induced.

Recently, it has been showed that recombinant proteins precipitated in aggregates could be re-solubilised in vivo. Aggregate re-folding was induced after that translation inhibition made available foldases and chaperones otherwise employed in metabolic folding (Carrió and Villaverde, 2001, FEBS Letts. 489, 29-33). We applied this idea to our system in which the accumulation of recombinant chaperones was possible.

The expression of coiled-coil Xklp3B was induced overnight at 0.5 mg/mL of arabinose (FIG. 17C, lane 1). The amount of recovered soluble protein was low and inhibited or almost completely prevented when chaperones (K+J+E+GroELS+ClpB) expression was IPTG-induced together or before arabinose addition (FIG. 17C, lanes 2 and 3). These data confirm the results collected using GTR1 and explained considering a competition among the recombinant proteins (FIG. 17B). In contrast, the removal of the arabinose-containing medium and the addition of fresh medium plus chloramphenicol had a positive effect on the amount of recovered soluble protein (FIG. 17C, lane 4). Apparently, the standard cellular folding machinery is, therefore, sufficient to partially re-fold the aggregated recombinant target protein. Nevertheless, a strong re-solubilisation improvement of the target protein was observed only when arabinose was removed, the pellet was re-suspended in fresh medium and chaperone-expression was induced by 0.2 mM IPTG addition (FIG. 17C, lane 5). A similar improvement at a slightly lower extent was obtained by the simple addition of a sufficiently high amount of IPTG (0.2 mM) to the arabinose-containing medium (FIG. 17C, lane 6). Therefore, it seems that it is possible to exploit the inhibitory effect of an overwhelming chaperone expression on the arabinose-regulated target protein to switch the system from Xklp3B to chaperone expression. Then, in conditions that inhibit the further expression of Xklp3B (comments to FIG. 17B), the available chaperones induce the re-folding of the already aggregated target protein without the need to remove the arabinose from the medium.

The collected results provide new information concerning the co-transformation of more than one recombinant proteins and confirm that chaperone co-transformation can increase the amount of soluble target protein. They also indicate that interactions among transformed plasmids and among corresponding proteins need to find an equilibrium in the host cell to optimise the co-transformation benefit. In fact, it seems that chaperones can somehow compete with the target protein, meaning that some care is required to optimise each candidate system, although this is well within the ambit of the skilled worker. Nevertheless, the reciprocal expression inhibition between target protein and chaperones can be exploited to tune the expression rate and improve the amount of soluble target protein. We must only be aware that the conditions need to be optimised since the accumulation rate is specific for each recombinant protein.

1. A method for the expression of a recombinant protein of interest, said method comprising:
a) culturing a host cell which expresses:
i) one or more genes encoding the recombinant protein(s) of interest;
ii) at least two genes encoding proteins selected from the group consisting of the chaperone proteins GroEL, GroES, DnaK, DnaJ, GrpE, ClpB and their homologs under conditions suitable for protein expression; and
b) separating said recombinant protein of interest from the host cell culture.

2. A method according to claim 1, wherein the genes selected in step a) ii) include DnaK, DnaJ and GrpE or homologs thereof.

3. A method according to claim 2, wherein the genes selected in step a) ii) additionally include ClpB or a homolog thereof.

4. A method according to claim 1, wherein the genes selected in step a) ii) include GroES and GroEL or homologs thereof.

5. A method according to claim 4, wherein the genes selected in step a) ii) include the DnaK, DnaJ, GrpE, ClpB, GroES and GroEL genes or homologs thereof.

6. A method for the expression of a recombinant protein of interest, said method comprising:
a) culturing under conditions suitable for protein expression a host cell which expresses:
i) one or more genes encoding one or more recombinant protein(s) of interest;
ii) one or more genes encoding proteins selected from the group consisting of the chaperone proteins GroEL, GroES, DnaK, DnaJ, GrpE, ClpB and their homologs;
iii) one or more genes encoding proteins selected from the group consisting of the small heatshock proteins of the IbpA family small heatshock proteins of the IbpB family and homologs thereof; and
(b) separating said recombinant protein of interest from the host cell culture.

7. A method according to claim 1 wherein the levels of the respective chaperone proteins are controlled.

8. A method according to claim 7, wherein said levels of chaperone proteins are controlled by expressing the genes encoding the respective chaperone proteins from different promoters.

9. A method according to claim 7, wherein the respective chaperone proteins are expressed using expression systems of different strength.

10. A method according to claim 7, wherein said chaperone proteins are over-expressed relative to the expression levels that occur naturally in non-recombinant cells.

11. A method according to claim 1, wherein the levels of the chaperone proteins relative to the recombinant protein(s) of interest are controlled by expressing the genes encoding the respective proteins from different promoters or by using different polymerases.

12. A method according to claim 1, wherein in culturing step a) of the method, a block in protein synthesis is imposed, for example, by the addition of an effective amount of a protein synthesis inhibitor to the culture system, once a desired level of recombinant protein of interest has accumulated.

13. A method according to claim 12, wherein the chosen protein synthesis inhibitor is chloramphenicol, tetracycline, gentamycin or streptomycin.

14. A method according to claim 1, wherein in culturing step a) of the method, a reduction in gene transcription is imposed, for example, by removal of any agents that are effective to induce recombinant protein expression, or via the addition of a transcription blocking compound, once a desired level of recombinant protein of interest has accumulated

15. A method for the expression of a recombinant protein of interest, said method comprising:
a) culturing a host cell which expresses:
i) one or more genes encoding the recombinant protein(s) of interest;
ii) one or more genes encoding one or more proteins selected from the group consisting of the chaperone proteins GroEL, GroES, DnaK, DnaJ, GrpE, ClpB and their homologs; under conditions suitable for protein expression;
b) imposing a block in protein synthesis, for example, by the addition of an effective amount of a protein synthesis inhibitor to the culture system, once a desired level of recombinant protein of interest has accumulated; and
c) separating said recombinant protein of interest from the host cell culture.

16. A method for the expression of a recombinant protein of interest, said method comprising:
a) culturing a host cell which expresses:
i) one or more genes encoding the recombinant protein(s) of interest;
ii) one or more genes encoding one or more proteins selected from the group consisting of the chaperone proteins GroEL, GroES, DnaK, DnaJ, GrpE, ClpB and their homologs; under conditions suitable for protein expression;
b) imposing a reduction in gene transcription, for example, by removal of any agents that are effective to induce recombinant protein expression, or via the addition of a transcription blocking compound, once a desired level of recombinant protein of interest has accumulated; and
c) separating said recombinant protein of interest from the host cell culture.

17. A method according to claim 15, wherein said host cells additionally expresses one or more genes encoding proteins selected from the group consisting of the small heatshock proteins of the IbpA family and/or the IbpB family and/or their homologs.

18. A method according claim 14, wherein in step a) ii), a combination of chaperone proteins is expressed.

19. A method according to claim 15, wherein the chosen protein synthesis inhibitor is chloramphenicol, tetracycline, gentamycin or streptomycin.

20. A method according to claim 1, wherein said cultured host cell is a prokaryotic cell, such as an E. coli cell, a Lactococcus cell, a Lactobacillus cell or a Bacillus subtilis cell, or a eukaryotic cell such as a yeast cell, for example a Pichia or Saccharomyces yeast cell, or an insect cell, for example after baculoviral infection.

21. A method according to claim 1, wherein an optimised yield of said recombinant protein of interest is manifested by increasing the level of de novo protein folding.

22. A method according to claim 1, wherein an optimised yield of said recombinant protein of interest is manifested by increasing the level of in vivo refolding of aggregated, or misfolded soluble, recombinant protein.

23. A method according to claim 1, wherein an optimised yield of said recombinant protein of interest is manifested by increasing the level of in vitro refolding of aggregated, or misfolded soluble, recombinant protein.

24. A method according to claim 20, wherein an optimised yield of said recombinant protein is manifested by increasing the level of de novo protein folding in combination with an increased level of in vivo protein refolding and/or in vitro protein refolding.

25. A method according to claim 21, wherein said increased level of folding or re-folding results in increased solubility of the recombinant protein of interest.

26. A method according to claim 21, wherein said increased level of folding or re-folding results in increased activity of the recombinant protein of interest.

27. A method for increasing the degree of refolding of a recombinant protein of interest, said method comprising adding a composition containing a chaperone protein to a preparation of the recombinant protein of interest in vitro.

28. A method according to claim 27, wherein a combination of chaperone proteins is added to the preparation of the recombinant protein of interest.

29. A method according to claim 27, wherein the preparation of the recombinant protein of interest is a preparation of soluble recombinant protein that has been precipitated in vivo.

30. A method according to claim 27, wherein the preparation of the soluble recombinant protein of interest is a preparation of in vitro precipitated recombinant protein.

31. A method according claim 27, wherein said composition containing the chaperone protein(s) is added after removal of any agents that are effective to induce soluble recombinant protein expression or after addition of a transcription blocking compound.

32. A method according to claim 27, additionally comprising the step of imposing a block in protein synthesis, such as by the addition of an effective amount of a protein synthesis inhibitor to the culture system.

33. A method according to claim 32, wherein the chosen protein synthesis inhibitor is chloramphenicol, tetracycline, gentamycin or streptomycin.

34. A method according claim 1, wherein the refolding temperature and time course of refolding are controlled.

35. A method according to claim 27, additionally comprising the use of one or more proteins selected from the group consisting of the small heatshock proteins of the IbpA family, the heatshock proteins of the IbpB family and homologs thereof.

36. The use of one or more genes encoding one or more proteins selected from the group consisting of the chaperone proteins GroEL, GroES, DnaK, DnaJ, GrpE, ClpB and their homologs, and one or more genes encoding proteins selected from the group consisting of the small heatshock proteins of the IbpA family, the small heatshock proteins of the IbpB family and homologs thereof, in the manufacture of a medicament for the treatment of disease in which the presence of aggregated proteins are implicated.

37. The use of one or more selected from the group consisting of the chaperone proteins GroEL, GroES, DnaK, DnaJ, GrpE, ClpB and their homologs, and one or more genes encoding proteins selected from the group consisting of the small heatshock proteins of the IbpA family, the small heatshock proteins of the IbpB family and homologs thereof, in the manufacture of a medicament for the treatment of disease in which the presence of aggregated proteins are implicated.

38. A method of treating a patient suffering from a disease in which the presence of aggregated proteins is implicated, comprising administering one or more genes encoding one or more proteins selected from the group consisting of the chaperone proteins GroEL, GroES, DnaK, DnaJ, GrpE, ClpB and their homologs, and one or more genes encoding proteins selected from the group consisting of the small heatshock proteins of the IbpA family, the small heatshock proteins of the IbpB family and homologs thereof.

39. A method of treating a patient suffering from a disease in which the presence of aggregated proteins is implicated, comprising administering one or more proteins selected from the group consisting of the chaperone proteins GroEL, GroES, DnaK, DnaJ, GrpE, ClpB and their homologs, and one or more proteins selected from the group consisting of the small heatshock proteins of the IbpA family, the small heatshock proteins of the IbpB family and homologs thereof.

40. The method of claim 38, wherein the disease is late or early onset Alzheimer's disease, SAA amyloidosis, hereditary Icelandic syndrome, multiple myeloma, or a spongiform encephalopathy.

41. A method according to claim 2, wherein the genes selected in step a) ii) include GroES and GroEL or homologs thereof

42. A method according to claim 3, wherein the genes selected in step a) ii) include GroES and GroEL or homologs thereof.

43. A method according to claim 41, wherein the genes selected in step a) ii) include the DnaK, DnaJ, GrpE, ClpB, GroES and GroEL genes or homologs thereof.

44. A method according to claim 42, wherein the genes selected in step a) ii) include the DnaK, DnaJ, GrpE, ClpB, GroES and GroEL genes or homologs thereof.

45. A method according to claim 16, wherein said host cells additionally expresses one or more genes encoding proteins selected from the group consisting of the small heatshock proteins of the IbpA family, the small heatshock proteins of the IbpB family and homologs thereof.

46. The method of claim 39, wherein the disease is late or early onset Alzheimer's disease, SAA amyloidosis, hereditary Icelandic syndrome, multiple myeloma, or a spongiform encephalopathy.
